(19) United States
(12) Patent Application Publication
(10) Pub. No.: US 2004/0101481 A1
(43) Pub. Date: May 27, 2004

(54) Acute treatment of headache with phenothiazine antipsychotics

(75) Inventors: Ron L. Hale, Woodside, CA (US); Peter M. Lloyd, Walnut Creek, CA (US); Amy T. Lu, Los Altos, CA (US); Patrik Munzar, Belmont, CA (US); Joshua D. Rabinowitz, Mountain View, CA (US); Roman Skowronski, Palo Alto, CA (US)

Correspondence Address: ALEXZA MOLECULAR DELIVERY CORPORATION, 1001 EAST MEADOW CIRCLE, PALO ALTO, CA, 94303, US

(73) Assignee: Alexza Molecular Delivery Corporation, Palo Alto, CA (US)

(21) Appl. No.: 10/719,763

(22) Filed: Nov. 20, 2003

Related U.S. Application Data
(60) Provisional application No. 60/429,404, filed on Nov. 26, 2002.

(51) Int. Cl. A61L 9/04

(52) U.S. Cl. 424/45

(57) ABSTRACT

Methods of treating headache with antipsychotics are provided. A kit for treating headache is also provided, comprising an antipsychotic and a device for rapid delivery of the antipsychotic.

### RELATED APPLICATIONS

This application claims the benefit of priority of U.S. Provisional Application No. 60/429,404, filed Nov. 26, 2002. Application No. 60/429,404 is incorporated by reference herein in its entirety for any purpose.

### FIELD OF THE INVENTION

The application discloses methods of treating a headache by administering an antipsychotic. The application also discloses kits for treating a headache.

### BACKGROUND OF THE INVENTION

A variety of compounds have been used in the preventative and/or acute treatment of various types of headache, including tension-type and migraine headache. A current compound, sumatriptan, is ineffective in treating many migraine headaches when given orally, and is associated with the life-threatening side effect of myocardial ischemia (heart attack). Two compounds that have been used in the treatment of even relatively refractory and severe headache are the phenothiazine antipsychotics prochlorperazine and chlorpromazine. These compounds are currently used in the treatment of headache at doses of generally at least 10 mg in an adult (0.15 mg/kg).

### SUMMARY OF CERTAIN EMBODIMENTS OF THE INVENTION

In certain embodiments, a method of treating a headache comprising administering by inhalation a composition comprising an antipsychotic to a patient in need of headache relief is provided.

In certain embodiments, a method of treating a headache, comprising administering by inhalation about 1 mg to 18 mg prochlorperazine to a patient in need of headache relief, wherein the prochlorperazine is administered such that the peak plasma concentration of the prochlorperazine is obtained within 15 minutes of initiation of administration of the prochlorperazine and wherein a decrease in headache severity is obtained within 2 hours of prochlorperazine administration, is provided.

In certain embodiments, a method of treating a migraine headache, comprising administering less than 9 mg of an antipsychotic to a patient in need of headache relief, wherein the peak plasma concentration of the antipsychotic is obtained within 15 minutes of initiation of administration of the antipsychotic, wherein a decrease in headache severity is obtained within 1 hour of initiation of administration of the antipsychotic, and wherein the decrease in headache severity persists for at least 12 hours after initiation of administration of the antipsychotic.

In certain embodiments, a kit for the treatment of headache comprising an antipsychotic and an inhalation delivery device is provided.

### BRIEF DESCRIPTION OF THE DRAWINGS

FIG. 1A shows a graph of time after termination of dosing (in hours) versus plasma concentration of prochlorperazine (in ng/mL) in dogs treated by inhalation with 12 mg/kg prochlorperazine for 10 minutes, as discussed in Example 1. FIG. 1B shows a graph of the same data as in FIG. 1A, but expanded to focus on the time period from initiation of treatment to 6.4 hours post treatment.

FIG. 2 shows a graph of dose of prochlorperazine (in mg) versus decrease in headache pain at 60 minutes (on a 4.0-point scale) in subjects treated intravenously with 0-10 mg prochlorperazine, as discussed in Example 2.

FIG. 3 shows a graph of dose of prochlorperazine (in mg) versus percent of patients free of pain at 1 hr, 4 hr, and 24 hr post initiation of intravenous administration of prochlorperazine, as discussed in Example 2.

FIG. 4 shows the preliminary results of an intravenous dose-ranging study of prochlorperazine, as discussed in Example 2. FIG. 4A shows a graph of time (in minutes) versus change in total pain severity from baseline (on a −2.0 scale) in subjects treated intravenously with 0-10 mg prochlorperazine. FIG. 4B shows a bar graph of percent of subjects free of pain at one hour and at two hours in subjects treated intravenously with 0-10 mg prochlorperazine. FIG. 4C shows a graph of time (in minutes) versus change in migraine pain severity from baseline (on a −2.0 scale) in subjects treated intravenously with 0-10 mg prochlorperazine. FIG. 4D shows a bar graph of percent of subjects free of migraine pain at one hour and at two hours in subjects treated intravenously with 0-10 mg prochlorperazine.

FIG. 5 shows a graph of purity of thermal vapor as a function of olanzapine film thickness, in micrometers, for olanzapine free base, as discussed in Example 9.

FIG. 6 shows a graph of purity of thermal vapor as a function of prochlorperazine film thickness, in micrometers, for prochlorperazine free base, as discussed in Example 10.

FIG. 7 shows a graph of purity of thermal vapor as a function of quetiapine film thickness, in micrometers, for quetiapine free base, as discussed in Example 13.

### DETAILED DESCRIPTION OF CERTAIN EXEMPLARY EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless specifically stated otherwise. Furthermore, the use of the term “including”, as well as other forms, such as “includes” and “included”, is not limiting. The use of the term “portion” may include part of a moiety or the entire moiety. Also, terms such as “element” or “component” encompass both elements and components comprising one unit and elements and components that comprise more than one subunit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Certain Definitions and Terms

The term “acetophenazine” refers to 1-[10-[3-[4-(2-Hydroxyethyl)-1-piperazinyl]propyl]-10H-phenothiazin-2-yl]ethanone.

The term “administering by inhalation” refers to the administration of a composition to a patient in aerosol form such that the patient inhales the composition by mouth or endotracheal tube in the pulmonary tract. “Administration by inhalation” does not include intranasal administration in this patent application. Intranasal administration will be specified separately from administration by inhalation.

The term “aerodynamic diameter” of a given particle refers to the diameter of a spherical droplet with a density of 1 g/mL (the density of water) that has the same settling velocity as the given particle.

The term “aerosol” refers to a suspension of solid or liquid particles in a gas. Exemplary nonlimiting aerosol preparations suitable for administration by inhalation to a patient include, but are not limited to, pure liquid droplets, solutions in liquid droplet form and solids in powder form. In certain embodiments, an aerosol preparation can include a pharmaceutically acceptable carrier. In certain embodiments, a pharmaceutically acceptable carrier is an inert compressed gas, e.g., nitrogen.

The term “amisulpride” refers to 4-amino-N-[(1-ethyl-2-pyrrolidinyl)methyl]-5-(ethylsulfonyl)-2-methoxybenzamide.

The term “amoxapine” refers to 2-chloro-1 1-(1-piperazinyl)dibenz[b,f] [1,4]oxazepine.

The term “antipsychotic” refers to compounds that are used in treatment of psychotic diseases, for example schizophrenia and other serious mental health diseases, or compounds that act at least in part to block the action of dopamine in the central nervous system of a mammal. Exemplary antipsychotics include, but are not limited to, acetophenazine, alizapride, amisulpride, amoxapine, amperozide, aripiprazole, benperidol, benzquinamide, bromperidol, buramate, butaclamol, butaperazine, carphenazine, carpipramine, chlorpromazine, chlorprothixene, clocapramine, clomacran, clopenthixol, clospirazine, clothiapine, clozapine, cyamemazine, droperidol, flupenthixol, fluphenazine, fluspirilene, haloperidol, iloperidone, loxapine, melperone, mesoridazine, metofenazate, molindone, perphenazine, pimozide, prochlorperazine, promethazine, olanzapine, penfluridol, pericyazine, pipamerone, piperacetazine, pipotiazine, promazine, remoxipride, risperidone, sertindole, spiperone, sulpiride, thiothixene, thioridazine, trifluoperazine, trifluperidol, ziprasidone, zotepine, and zuclopenthixol.

The term “antipsychotic degradation product” refers to a compound resulting from a chemical modification of the antipsychotic during an antipsychotic vaporization-condensation process. In certain embodiments, the modification can be the result of a thermally or photochemically induced reaction. Exemplary thermally- or photochemically-induced reactions include, but are not limited to, oxidation and hydrolysis.

The term “aripiprazole” refers to 7-[4-[4-(2,3-Dichlorophenyl)-1-piperazinyl]butoxy]-3,4-dihydro-2(1H)-quinolinone.

The term “a typical antipsychotic” refers to a subset of classical antipsychotics consisting of olanzapine, clozapine, risperidone, quetiapine, sertindole, ziprasidone, and zotepine.

The term “a typical-like antipsychotics” refers to a subset of the classical antipsychotics consisting of classical antipsychotics wherein the classical antipsychotic has at least 7 times greater affinity for 5HT2A serotonin receptors than for D2 dopamine receptors.

The term “baseline” refers to a level of headache pain in a subject at the time treatment is initiated. In certain embodiments, the headache pain at baseline is moderate to severe.

The term “chlorpromazine” refers to 10-(3-dimethylaminopropyl)-2-chlorphenothiazine.

The term “chlorprothixene” refers to (Z)-3-(2-chloro-9H-thioxanthen-9-ylidene)-N,N-dimethyl-1-propanamine.

The term “classical antipsychotics” refers to antipsychotics that act at least in part to block the action of dopamine in the central nervous system of a mammal.

The term “clozapine” refers to 8-chloro-11-(4-methyl-1-piperazinyl)-5H-dibenzo[b,e][1,4]diazepine.

The term “decrease,” when referring to a decrease in headache severity, refers to a lessening of headache pain when comparing headache severity in patients treated with an antipsychotic to headache severity in patients treated with a placebo or to patients not treated. In certain embodiments, the lessening is statistically significant, e.g., having a P<0.05.

The term “dose” refers to a quantity of an antipsychotic which is administered to a patient in need of headache relief.

The term “droperidol” refers to 1-[1-[4-(4-fluorophenyl)-4-oxobutyl]-1,2,3,6-tetrahydro-4-pyridinyl]-1,3-dihydro-2H-benzimidazol-2-one.

The term “effective human therapeutic dose” refers to the amount of an antipsychotic that achieves the desired effect or efficacy. In certain embodiments, the desired effect or efficacy can be an abatement of symptoms. In certain embodiments, the desired effect or efficacy can be a cessation of an episode.

The term “flupenthixol” refers to 4-[3-[2-(trifluoromethyl)-9H-thioxanthen-9-ylidene]propyl]-1-piperazineethanol.

The term “fluphenazine” refers to 4-[3-[2-(trifluoromethyl)-10H-phenothiazin-10-yl]propyl]-1-piperazine-ethanol.

The term “fraction of antipsychotic” refers to the quantity of antipsychotic present in the aerosol particles divided by the quantity of antipsychotic plus antipsychotic degradation product present in the aerosol, i.e. (quantity of antipsychotic present in the aerosol particles)/((quantity of antipsychotic present in the aerosol)+(sum of quantities of all antipsychotic degradation products present in the aerosol)). The term “percent antipsychotic” refers to the fraction of antipsychotic multiplied by 100%.

The term “fraction antipsychotic degradation product” refers to the quantity of antipsychotic degradation products present in the aerosol particles divided by the quantity of antipsychotic plus antipsychotic degradation product present in the aerosol, i.e. (sum of quantities of all antipsychotic degradation products present in the aerosol)/((quantity of antipsychotic present in the aerosol)+(sum of quantities of all antipsychotic degradation products present in the aerosol)). The term “percent antipsychotic degradation product” refers to the fraction of antipsychotic degradation product multiplied by 100%, whereas “purity” of the aerosol refers to 100% minus the percent antipsychotic degradation products. To determine the percent or fraction antipsychotic degradation product, in certain embodiments, the aerosol is collected in a trap. Exemplary traps include, but are not limited to, a filter, glass wool, an impinger, a solvent trap, and a cold trap. In certain embodiments, the trap is then extracted with a solvent, e.g. acetonitrile, and the extract subjected to analysis by any of a variety of analytical methods known in the art. In certain embodiments, gas or liquid chromatography is used. An exemplary nonlimiting type of liquid chromatography is high performance liquid chromatography.

The term “given interval of time” refers to a period of time in which an administered antipsychotic is expected to have a therapeutic effect, and/or the amount of time it takes for the antipsychotic to reach or to approximately reach peak plasma concentrations.

The term “haloperidol” refers to 4-[4-(4-chlorophenyl)-4-hydroxy-1-piperidinyl]-1-(4-fluorophenyl)-1-butanone.

The term “headache” refers to a condition of mild to severe pain associated with the head, and also includes upper back or neck pain. Exemplary varieties of headaches include, but are not limited to, migraine headache, tension-type headache, and cluster headache.

The term “headache free” refers to a patient suffering from a headache who, after initiation of administration of an antipsychotic, no longer has a headache. In certain embodiments, a patient's score of 5 on a categorical headache pain relief scale (where a score of 1 indicates no pain relief, a score of 2 indicates some pain relief, a score of 3 indicates moderate pain relief, a score of 4 indicates much pain relief, and a score of 5 indicates complete pain relief) indicates that a patient is headache free. In other embodiments, a patient's score of 0 on a standard categorical 4-point headache severity scale (where a score of 0 indicates absence of headache, a score of 1 indicates mild headache, a score of 2 indicates moderate headache, and a score of 3 indicates severe headache) indicates that a patient is headache free.

The term “headache relief” refers to a decrease in the level of pain suffered by a patient with a headache after initiation of administration of antipsychotic to the patient. In certain embodiments, a patient's score on a categorical headache severity scale (where a score of 0 indicates absence of headache, a score of 1 indicates mild headache, a score of 2 indicates moderate headache, and a score of 3 indicates severe headache) which is lower than the patient's score before initiation of administration of an antipsychotic indicates that the patient is experiencing headache relief. In other embodiments, a patients score of 2 or 3 or 4 or above on a categorical headache pain relief scale (where a score of 1 indicates no pain relief, a score of 2 indicates some pain relief, a score of 3 indicates moderate pain relief, a score of 4 indicates much pain relief, and a score of 5 indicates complete pain relief) indicates that the patient is experiencing headache relief.

The term “iloperidone” refers to 1-[4-[3-[4-(6-fluoro-1,2-benzisoxazol-3-yl)-1-piperidinyl]propoxy]-3-methoxyphenyl]ethanone.

The term “intranasal administration” refers to the administration of an antipsychotic to a patient by an intranasal route.

The term “loxapine” refers to 2-chloro-11-(4-methyl-1-piperazinyl)dibenz[b,f][1,4]oxazepine.

The term “mass median aerodynamic diameter” or “MMAD” of an aerosol refers to the aerodynamic diameter for which half the particulate mass of the aerosol is contributed by particles with an aerodynamic diameter larger than the MMAD and half by particles with an aerodynamic diameter smaller than the MMAD.

The term “melperone” refers to 1-(4-fluorophenyl)-4-(4-methyl-1-piperidinyl)-1-butanone.

The term “mesoridazine” refers to 10-[2-(1-Methyl-2-piperidinyl)ethyl]-2-(methylsulfinyl)-10H-phenothiazine.

The term “molindone” refers to 3-ethyl-1,5,6,7-tetrahydro-2-methyl-5-(4-morpholinylmethyl)-4H-indol-4-one.

The term “non-phenothiazine antipsychotic” refers to a subset of antipsychotics that do not contain a phenothiazine structure. In certain embodiments, the non-phenothiazine antipsychotic is a typical non-phenothiazine antipsychotic or a typical-like non-phenothiazine antipsychotic. In certain embodiments, the non-phenothiazine antipsychotic is an a typical non-phenothiazine antipsychotic. Exemplary non-phenothiazine antipsychotics include, but are not limited to, amisulpride, aripiprazole, chlorprothixene, clozapine, droperidol, flupenthixol, haloperidol, iloperidone, loxapine, melperone, molindone, pimozide, olanzapine, remoxipride, risperidone, thiothixene, ziprasidone, zotepine, and zuclopenthixol.

The term “olanzapine” refers to 2-methyl-4-(4-methyl-1-piperazinyl)-10H-thieno [2,3-b][1,5]benzodiazepine.

The term “peak plasma concentration” refers to the maximum level of the antipsychotic obtained in the plasma of a patient after initiation of administration of the antipsychotic to the patient.

The term “perphenazine” refers to 4[3(2-chloro-10H-phenothiazin-10-yl)propyl]-1-piperazine-ethanol.

The term “phenothiazine antipsychotic” refers to a classical antipsychotic that contains a phenothiazine structure. Exemplary phenothiazine antipsychotics include, but are not limited to, prochlorperazine, trifluoperazine, fluphenazine, promethazine, perphenazine, chlorpromazine, and thioridazine, mesoridazine, and acetophenazine.

The term “phenothiazine structure” refers to a heterocyclic structure comprising a central 1,4-thiazine six-membered ring with two additional six-membered aromatic carbon rings symmetrically joined at the 1,3- and 5,6-positions. Typically phenothiazine antipsychotics with the phenothiazine structure are substituted at N-10 by a chain having a terminal tertiary amine group 2-3 atoms distant.

The term “pimozide” refers to 1-[1-[4,4-bis(4-fluorophenyl)butyl]-4-piperidinyl]-1,3-dihydro-2H-benzimidazol-2-one.

The term “prochlorperazine” refers to 2-chloro-10-[3-(4-methyl-1-piperazinyl-)propyl]-10H-phenothiazine.

The term “promethazine” refers to 10-(2-dimethylaminopropyl)-phenothiazine.

The term “remoxipride” refers to 3-bromo-N-[[(2S)-1-ethyl-2-pyrrolidinyl]methyl]-2,6-dimethoxybenzamide.

The term “risperidone” refers to 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one.

The term “self-administer” or “self-administration” refers to a patient administering one or more doses of a drug without assistance from a medical professional. The route of self-administration may be any medically acceptable route of drug delivery. Exemplary routes of drug delivery include, but are not limited to, intranasally, intramuscularly, intravenously, orally, parenterally, transdermally, rectally, and by inhalation.

The term “sertindole” refers to 1-[2-[4-[5-chloro-1-(4-fluorophenyl)-1H-indol-3-yl]-1-piperidinyl] ethyl]-2-imidazolidinone.

The term “statistically significant compared to baseline” refers to the case wherein a measurement in one or more patients taken at a particular time point following initiation of treatment is statistically significantly different from the same measurement in the one or more patients prior to treatment as indicated by a p-value of 0.05 when the two sets of measurements are compared using an appropriate statistical test.

The term “statistically significant compared to placebo” refers to the case wherein a measurement in one or more patients treated with drug is statistically significantly different from the same measurement in one or more patients treated with placebo as indicated by a p-value of 0.05 when the two sets of measurements are compared using an appropriate statistical test.

The term “therapeutic systemic concentration” refers to the concentration of an antipsychotic within the bloodstream of a patient at which a therapeutic effect of the antipsychotic is achieved. An exemplary nonlimiting therapeutic systemic concentration is the concentration of an antipsychotic within the bloodstream of a patient at which a decrease in headache severity is obtained.

The term “thermal vapor” refers to an aerosol, to a vapor phase, or to a mixture of an aerosol and a vapor phase. In certain embodiments, the thermal vapor is formed by heating. In certain embodiments, the thermal vapor comprises a drug. In certain embodiments, the thermal vapor comprises a drug and a carrier. The term “vapor phase” refers to a gaseous phase.

The term “thioridazine” refers to 10-[2-(1-methyl-2-piperidinyl)ethyl]-2-(methylthio)-10H-phenothiazine.

The term “thiothixene” refers to N,N-dimethyl-9-[3-(4-methyl-1-piperazinyl)propylidene]thioxanthene-2-sulfonamide.

The term “trifluoperazine” refers to 2-trifluoro-methyl-10-[3′-(1-methyl-4-p-piperazinyl)-propyl]phenothiazine.

The term “typical antipsychotic” refers to antipsychotics that are classical antipsychotics excluding a typical antipsychotics.

The term “typical non-phenothiazine antipsychotic” refers to typical antipsychotics excluding phenothiazine antipsychotics. Exemplary typical non-phenothiazine antipsychotics include, but are not limited to, chlorprothixene, droperidol, flupenthixol, haloperidol, loxapine, melperone, molindone, pimozide, thiothixene, and zuclopenthixol.

The term “ziprasidone” refers to 5-[2-[4-(1,2-benzisothiazol-3-yl)-1-piperazinyl]ethyl]-6-chloro-1,3-dihydro-2H-indol-2-one.

The term “zotepine” refers to 2-[(8-chlorodibenzo[b,f]thiepin-10-yl)oxy]--N,N-dimethylethanamine.

The term “zuclopenthixol” refers to 4-[(3Z)-3-(2-chloro-9H-thioxanthen-9-ylidene)propyl]-1-piperazineethanol.

Certain Embodiments of the Invention

Method Embodiments

In certain embodiments, methods of treating a headache comprising administering by inhalation a composition comprising an antipsychotic to a patient in need of headache relief are provided.

In certain embodiments, the antipsychotic is selected from acetophenazine, alizapride, amisulpride, amoxapine, amperozide, aripiprazole, benperidol, benzquinamide, bromperidol, buramate, butaclamol, butaperazine, carphenazine, carpipramine, chlorpromazine, chlorprothixene, clocapramine, clomacran, clopenthixol, clospirazine, clothiapine, clozapine, cyamemazine, droperidol, flupenthixol, fluphenazine, fluspirilene, haloperidol, iloperidone, loxapine, melperone, mesoridazine, metofenazate, molindone, perphenazine, pimozide, prochlorperazine, promethazine, olanzapine, penfluridol, pericyazine, pipamerone, piperacetazine, pipotiazine, promazine, remoxipride, risperidone, sertindole, spiperone, sulpiride, thiothixene, thioridazine, trifluoperazine, trifluperidol, ziprasidone, zotepine, and zuclopenthixol.

In certain embodiments, the antipsychotic is a phenothiazine antipsychotic. In certain embodiments, the phenothiazine antipsychotic is selected from prochlorperazine, trifluoperazine, fluphenazine, promethazine, perphenazine, chlorpromazine, and thioridazine, mesoridazine, and acetophenazine. In certain embodiments, the antipsychotic is selected from prochlorperazine, trifluoperazine, fluphenazine, and perphenazine. In certain embodiments, the antipsychotic is prochlorperazine. In certain embodiments, prochlorperazine is administered by inhalation. In certain embodiments, the inhalation of prochlorperazine has no sustained effect on bronchoconstriction. In certain embodiments, two or more phenothiazine antipsychotics are combined.

In certain embodiments, the dose of phenothiazine antipsychotic administered to a patient in order to treat a headache is substantially lower than phenothiazine antipsychotic doses previously used in the art in the treatment of headaches. In certain embodiments, the dose of phenothiazine antipsychotic for administration by inhalation is about 0.1 mg to 5 mg of fluphenazine or trifluoperazine. In certain embodiments, the dose of phenothiazine antipsychotic for administration by inhalation is 0.1 mg, 0.25 mg, 0.5 mg, 0.75 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, or 5 mg of fluphenazine or trifluoperazine. In certain embodiments, the dose of phenothiazine antipsychotic for administration by inhalation is about 3 mg to 40 mg of chlorpromazine, thioridazine, or mesoridazine. In certain embodiments, the dose of phenothiazine antipsychotic is 3 mg, 5 mg, 7.5 mg, 10 mg, 12.5 mg, 15.0 mg, 17.5 mg, 20 mg, 22.5 mg, 25 mg, 27.5 mg, 30 mg, 32.5 mg, 35 mg, 37.5 mg, or 40 mg of chlorpromazine, thioridazine, or mesoridazine. In certain embodiments, the dose of phenothiazine antipsychotic for administration by inhalation is about 0.5 mg to 18 mg of prochlorperazine, perphenazine, acetophenazine, or promethazine. In certain embodiments, the dose of phenothiazine antipsychotic for administration by inhalation is 0.5 mg, 1 mg, 1.25 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, or 18 mg of prochlorperazine, perphenazine, acetophenazine, or promethazine. In certain embodiments, the dose of phenothiazine antipsychotic for intravenous administration is about 1 to 9 mg of prochlorperazine. In certain embodiments, the dose of phenothiazine antipsychotic for intravenous administration is about 1 to 5 mg of prochlorperazine. In certain embodiments, the dose of phenothiazine antipsychotic for intravenous administration is 0.5 mg, 1 mg, 1.25 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, or 9 mg of prochlorperazine.

In certain embodiments, the phenothiazine antipsychotic is prochlorperazine administered by inhalation at a dosage of about 1 to 18 mg. Bowden et al., Clin. Exp. Pharmacol. Physiol. 15(6): 457-463 (1988), reported that inhalation of 10 mg/mL of the phenothiazine antipsychotic trifluoperazine for the treatment of asthma gave rise to a significant bronchioconstrictive effect in patients treated with that antipsychotic. In certain embodiments, inhalation of the antipsychotic does not result in substantial bronchioconstriction.

In certain embodiments, the antipsychotic is a typical non-phenothiazine antipsychotic. In certain embodiments, the typical non-phenothiazine antipsychotic is selected from amisulpride, aripiprazole, chlorprothixene, droperidol, flupenthixol, haloperidol, iloperidone, loxapine, melperone, molindone, pimozide, remoxipride, thiothixene, and zuclopenthixol. In certain embodiments, two or more typical non-phenothiazine antipsychotics are combined.

In certain embodiments, the dose of the typical non-phenothiazine antipsychotic administered to a patient in need of headache relief is 50 mg or less. In certain embodiments, the dose of the typical non-phenothiazine antipsychotic for administration by inhalation is about 0.1 to 10 mg haloperidol, iloperidone, droperidol, or pimozide. In certain embodiments, the dose of the typical non-phenothiazine antipsychotic for administration by inhalation is 0.1 mg, 0.25 mg, 0.5 mg, 0.75 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.25 mg, 5.5 mg, 5.75 mg, 6 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg, or 10 mg of haloperidol, iloperidone, droperidol, or pimozide. In certain embodiments, the dose of the typical non-phenothiazine antipsychotic for administration by inhalation is 1 mg to 25 mg of aripiprazole, loxapine, molindone, thiothixene, flupenthixol, zuclopenthixol, or zotepine. In certain embodiments, the dose of the typical non-phenothiazine antipsychotic for administration by inhalation is 1 mg, 1.25 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, or 25 mg of aripiprazole, loxapine, molindone, thiothixene, flupenthixol, zuclopenthixol, or zotepine. In certain embodiments, the dose of the typical non-phenothiazine antipsychotic for administration by inhalation is about 3 mg to 50 mg of amisulpride, chlorprothixene, remoxipride or melperone. In certain embodiments, the dose of the typical non-phenothiazine antipsychotic for administration by inhalation is 3 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, or 50 mg of amisulpride, chlorprothixene, remoxipride or melperone.

In certain embodiments, the antipsychotic is an a typical non-phenothiazine antipsychotic. In certain embodiments, the a typical antipsychotic is selected from clozapine, olanzapine, quetiapine, risperidone, sertindole, ziprasidone, and zotepine. In certain embodiments, two or more a typical non-phenothiazine antipsychotics are combined.

In certain embodiments, the dose of the a typical non-phenothiazine antipsychotic administered to a patient in need of headache relief is 50 mg or less. In certain embodiments, the dose of the a typical non-phenothiazine antipsychotic for administration by inhalation is about 0.1 mg to 10 mg of olanzapine or risperidone. In certain embodiments, the dose of the a typical non-phenothiazine antipsychotic for administration by inhalation is 0.1 mg, 0.25 mg, 0.5 mg, 0.75 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.25 mg, 5.5 mg, 5.75 mg, 6 mg, 6.5 mg, 6.75 mg, 7 mg, 7.25 mg, 7.5 mg, 7.75 mg, 8 mg, 8.25 mg, 8.5 mg, 8.75 mg, 9 mg, 9.25 mg, 9.5 mg, 9.75 mg, or 10 mg of olanzapine or risperidone. In certain embodiments, the dose of the a typical non-phenothiazine antipsychotic for administration by inhalation is about 1 mg to 25 mg of sertindole, zotepine or ziprasidone. In certain embodiments, the dose of the a typical non-phenothiazine antipsychotic for administration by inhalation is 1 mg, 1.25 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, or 25 mg of sertindole, zotepine or ziprasidone. In certain embodiments, the dose of the a typical non-phenothiazine antipsychotic for administration by inhalation is about 3 mg to 50 mg of quetiapine or clozapine. In certain embodiments, the dose of the a typical non-phenothiazine antipsychotic for administration by inhalation is 3 mg, 5 mg, 7.5 mg, 10 mg, 12.5 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, or 50 mg of quetiapine or clozapine.

In certain embodiments, the headache to be treated is selected from at least one of a migraine headache, a tension-type headache, and a cluster headache. In certain embodiments, the headache to be treated is a combination of two or more of a migraine headache, a tension-type headache, and a cluster headache. In certain embodiments, the headache is of a nonspecific type. In certain embodiments, the headache arises from upper back or neck pain.

In certain embodiments, the antipsychotic is administered via any medically acceptable route of drug delivery. Exemplary nonlimiting routes of drug delivery include, but are not limited to, intranasally, intramuscularly, intravenously, orally, parenterally, transdermally, and rectally.

In certain embodiments, the antipsychotic is administered orally. Exemplary nonlimiting ways to accomplish oral administration of the antipsychotic include, but are not limited to, tablets, effervescent tablets, capsules, granulates, and powders. In certain embodiments, pharmacologically active ingredients are mixed with an inert solid diluent. Exemplary inert solid diluents include, but are not limited to, calcium carbonate, calcium phosphate and kaolin. In certain embodiments, the antipsychotic is provided in the form of soft gelatin capsules wherein the active ingredients are mixed with an oleaginous medium, e.g., but not limited to, liquid paraffin or olive oil. In certain embodiments, the antipsychotic is administered topically by mouth. Exemplary nonlimiting ways to accomplish topical administration include, but are not limited to, buccal tablets, sublingual tablets, drops, and lozenges.

In certain embodiments, the antipsychotic is administered by injection. Exemplary nonlimiting types of injection of the antipsychotic include, but are not limited to, intravenous injection, intramuscular injection, and subcutaneous injection, for example by bolus injection or continuous intravenous infusion. In certain embodiments, formulations for injection may be presented in unit dosage form, e.g., in ampoules or in multi-dose containers, with or without one or more added preservatives. In certain embodiments, formulations for injection can take such forms as suspensions, solutions, or emulsions in oily or aqueous vehicles, and may contain formulatory agents such as suspending, stabilizing, and/or dispersing agents. In certain embodiments, the active ingredient may be in powder form for dilution with a suitable vehicle, e.g., sterile pyrogen-free water, before use.

In certain embodiments, the antipsychotic may be formulated in rectal compositions such as suppositories or retention enemas, e.g., containing certain conventional suppository bases such as cocoa butter or other glyceride.

In certain embodiments, the antipsychotic is administered by inhalation. In certain embodiments, administration by inhalation results in rapid drug absorption without the need for injection. In certain embodiments, the administration by inhalation of the antipsychotic is performed by administration of a composition to a patient in aerosol form such that the patient inhales the composition by mouth or endotracheal tube in the pulmonary tract. In certain embodiments, administration by inhalation is accomplished using an inhalation delivery device. In certain embodiments, administration by inhalation is accomplished using Staccato™ Prochlorperazine for Inhalation. Non-limiting exemplary inhalation delivery devices include, but are not limited to, nebulizers, metered-dose inhalers, dry-powder inhalers or other inhalers known to those skilled in the art.

Nonlimiting exemplary inhalation devices are disclosed, e.g., in U.S. patent application Ser. Nos. 10/633,876 and U.S. Ser. No. 10/633,877, both filed on Aug. 4, 2003. Certain exemplary devices comprise a heat-conductive substrate onto which a film of antipsychotic is deposited. In certain embodiments, the surface area of the substrate is sufficient to yield a therapeutic dose of the antipsychotic aerosol when used by a subject. In certain embodiments, the desired dosage and selected antipsychotic film thickness dictate the minimum optimal substrate area in accord with the following relationship: film thickness (cm)×antipsychotic density (g/cm³)×substrate area (cm²)=dose (g). In certain embodiments, the calculated substrate area for a 5 mg dose of prochlorperazine is about 2.5 to 500 cm², and the film thickness is about 0.1 to 20 μm.

Certain heat-conductive materials for use in forming the substrate, according to certain embodiments, are known. Exemplary nonlimiting heat-conductive materials include, but are not limited to, metals, alloys, ceramics, and filled polymers. In various embodiments, the heat-conductive substrate can be of any geometry. In certain embodiments, the heat-conductive substrate has a surface with relatively few or substantially no surface irregularities so that a molecule of an antipsychotic vaporized from a film of the antipsychotic on the surface is unlikely to acquire sufficient energy to decompose through contact with (i) other hot vapor molecules, (ii) hot gases surrounding the area, and/or (iii) the substrate surface. In certain embodiments, when a molecule of an antipsychotic vaporized from a film of the antipsychotic on the surface does not acquire sufficient energy to result in cleavage of chemical bonds, decomposition of the antipsychotic is decreased. In certain embodiments, a rapid increase in velocity gradient of gases over the surface results in minimization of the hot gas region above the heated surface and decreases the time of transition of the vaporized antipsychotic to a cooler environment. Exemplary nonlimiting substrates are those that have impermeable surfaces or have an impermeable surface coating, including, but not limited to, metal foils, smooth metal surfaces, and non-porous ceramics.

In certain embodiments, the film of antipsychotic deposited on the substrate has a thickness of between about 0.05 μm and 20 μm. In certain embodiments, the film thickness for a given antipsychotic is such that antipsychotic-aerosol particles, formed by vaporizing the antipsychotic by heating the substrate and entraining the vapor in a gas stream, have (i) 10% by weight or less antipsychotic-degradation product, and (ii) at least 50% of the total amount of antipsychotic contained in the film. In certain instances, thinner antipsychotic films result in purer antipsychotic particles than thicker antipsychotic films. In certain embodiments, the structure and/or form of the antipsychotic are adjusted to increase aerosol purity and/or yield. In certain embodiments, the thermal vapor is produced in an inert atmosphere, e.g., in an inert gas such as argon, nitrogen, helium, or the like, to increase aerosol purity and/or yield. In certain embodiments, altered forms of the antipsychotic are used, e.g., a prodrug, a free base, free acid, or salt form, which impacts the purity and/or yield of the aerosol obtained.

Exemplary nonlimiting methods of deposition of an antipsychotic onto a substrate include, but are not limited to, (i) preparing a solution of antipsychotic in solvent, applying the solution to the exterior surface of the substrate, and removing the solvent to leave a film of antipsychotic, (ii) applying the antipsychotic to the substrate by dipping the substrate into an antipsychotic solution or by spraying, brushing, or otherwise applying the solution to the substrate, and (iii) preparing a melt of the antipsychotic and applying it to the substrate.

In certain embodiments, an inhalation delivery device includes a heating element incorporated into a solid substrate. In certain embodiments, an inhalation delivery device includes a heating element inserted into a hollow space of a hollow substrate. Exemplary nonlimiting heating elements include, but are not limited to, an electrical resistive wire that produces heat when a current flows through the wire, solid chemical fuel, chemical components that undergo an exothermic reaction, and inductive heat. In certain embodiments, a substrate is heated by conductive heating. In certain embodiments, substrate heating can be actuated by a user-activated mechanism on the housing of the inhalation delivery device, or by breath actuation. Certain non-limiting exemplary activation mechanisms are known in the art. In certain embodiments, an inhalation delivery device further comprises a power supply source and valving, if appropriate.

In certain embodiments, a heat source is effective to supply heat to a substrate at a rate that achieves a substrate temperature of at least about 200° C. In certain embodiments, a substrate temperature is about 200° C. to 500° C. Exemplary nonlimiting substrate temperatures include, but are not limited to, about 200° C., about 250° C., about 300° C., about 350° C., about 400° C., about 450° C., or about 500° C. In certain embodiments, the temperature used produces substantial volatilization of the antipsychotic from the substrate within about 0.5 to 2 seconds.

In certain embodiments, an inhalation delivery device includes a gas-flow control valve for limiting gas-flow rate through the condensation region to the selected gas-flow rate. For example, in certain embodiments, a gas-flow control valve limits airflow through the chamber as air is drawn by the user's mouth into and through the chamber. In certain embodiments, an inhalation delivery device includes one or more additional valves to control the total volumetric airflow through the device. In certain embodiments, the gas-flow control valve acts to limit air drawn into the device to a preselected level, e.g., about 15 L/min, corresponding to a selected airflow rate for producing aerosol particles of a selected size. In certain embodiments, once the selected airflow level is achieved, additional air drawn into the device creates a pressure drop across a bypass valve which then accommodates airflow through the bypass valve into the end of the device adjacent to the user's mouth.

In certain embodiments, a gas-flow control valve and one or more bypass valves may be used to control the gas velocity through the substrate chamber and hence to control the particle size of the aerosol particles produced by vapor condensation. In certain embodiments, the particle size distribution of the aerosol is determined by the concentration of the antipsychotic. In certain embodiments, smaller or larger particles of the antipsychotic may be obtained by altering the gas velocity through the condensation region of the substrate chamber. In certain embodiments, condensation particles in the size range of about 1 μm to 3.5 μm MMAD are produced by use of a condensation chamber with substantially smooth-surfaced walls and a gas-flow rate in the range of about 4 L/min to 50 L/min. In certain embodiments, particle size may be altered by modifying the cross-section of the substrate chamber condensation region to increase or decrease linear gas velocity for a given volumetric flow rate. In certain embodiments, particle size may be altered by the presence or absence of structures that produce turbulence within the chamber.

In certain embodiments, the bioavailability of thermal vapor ranges from about 20% to 100% of the amount of the antipsychotic subjected to thermal vaporization. In certain embodiments, the bioavailability of thermal vapor is in the range of 50-100% relative to the bioavailability of antipsychotics infused intravenously. In certain embodiments, the potency of the thermal vapor antipsychotic per unit plasma antipsychotic concentration is equal to or greater than that of the antipsychotic delivered by other routes of administration. In certain embodiments, thermal vapor delivery results in increased antipsychotic concentration in a target organ such as the brain, relative to the plasma antipsychotic concentration. For example, Lichtman et al., The Journal of Pharmacology and Experimental Therapeutics 279:69-76 (1996), discussed work that suggested that opiods administered by inhalation may have increased potency compared to those administered intravenously due to increased accessibility to the brain. In certain embodiments, the unit dose amount of an antipsychotic in thermal vapor form is similar to or less than a standard oral or intravenous dose.

In certain embodiments, determination of an appropriate dose of thermal vapor to be used to treat a headache can be performed via animal experiments and/or a dose-finding (Phase I/II) clinical trial. In certain embodiments, measurements of plasma antipsychotic concentrations after exposure of a test animal to an antipsychotic thermal vapor are made. See a non-limiting example discussed in Example 1. In certain embodiments, animal experiments may also be used to evaluate possible pulmonary toxicity of the thermal vapor. Because accurate extrapolation of animal experiment results to humans is facilitated if the test animal has a respiratory system similar to humans, mammals such as dogs or primates are useful test animals. See a non-limiting example discussed in Example 1. In certain embodiments, animal experiments may also be used to monitor behavioral or physiological responses in mammals. In certain embodiments, initial dose levels for testing in humans will generally be less than or equal to the least of the following doses: current standard intravenous dose, current standard oral dose, dose at which a physiological or behavioral response was obtained in the mammal experiments, and dose in the mammal model which resulted in plasma antipsychotic levels associated with a therapeutic effect of the antipsychotic in humans. In certain embodiments, dose escalation may then be performed in humans, until either an optimal therapeutic response is obtained or dose-limiting toxicity is encountered.

In certain embodiments, the antipsychotic compound is delivered as an aerosol. In certain embodiments, the mass median aerodynamic diameter (MMAD) of the aerosol particles is less than about 5 μm. In certain embodiments, the MMAD of the aerosol particles is less than about 3 μm. In certain embodiments, the MMAD is within a range of about 1 to 5 μm.

In certain embodiments, the composition comprising the antipsychotic further comprises a diluent appropriate for human administration. In certain embodiments, the diluent is water, saline, ethanol, propylene glycol, glycerol, or mixtures thereof.

In certain embodiments, the antipsychotic is delivered as a single compound. In certain embodiments, more than one antipsychotic are used in a composition or are separately administered. In certain embodiments, the antipsychotic is used in a composition or separately administered with one or more additional compounds utilized in pain management. Non-limiting exemplary compounds utilized in pain management include, but are not limited to, non-steroidal anti-inflammatory drugs, opioids, psychostimulants, barbiturates, benzodiazepines, and other compounds known to those skilled in the art.

In certain embodiments, the actual effective amount of antipsychotic for a particular patient can vary according to at least one of the specific antipsychotic or combination of antipsychotics being utilized; the particular composition formulated; the mode of administration; the age, weight, and condition of the patient; and the severity of the episode being treated.

In certain embodiments, the patient in need of headache relief is an animal. In certain embodiments, the animal is a mammal. In certain embodiments, the patient in need of headache relief is a human patient.

In certain embodiments, the antipsychotic is delivered by a route of administration that results in peak plasma concentrations in the patient being obtained rapidly after initiation of administration of the antipsychotic to the patient. In certain embodiments, the peak plasma concentration is obtained within 20 minutes after initiation of antipsychotic administration. In certain embodiments, the peak plasma concentration is obtained within 15 minutes after initiation of antipsychotic administration. In certain embodiments, the peak plasma concentration is obtained within 1 minute, 2 minutes, 3 minutes, 5 minutes, 10 minutes, 15 minutes, or 30 minutes of initiation of administration of the antipsychotic.

In certain embodiments, the concentration of antipsychotic in the plasma of the patient is at least 30% of the peak plasma concentration within 2 minutes of initiation of administration by inhalation. In certain embodiments, the concentration of antipsychotic in the plasma of the patient is at least 30% of the peak plasma concentration within 1 minute, 2 minutes, 3 minutes, 5 minutes, 10 minutes, 15 minutes, or 30 minutes of initiation of administration by inhalation.

In certain embodiments, the antipsychotic is delivered by a route of administration that results in a therapeutic systemic concentration of the antipsychotic in the patient being obtained rapidly after initiation of administration of the antipsychotic to the patient. In certain embodiments, the therapeutic systemic concentration of the antipsychotic is obtained within 30 minutes of initiation of administration. In certain embodiments, the therapeutic systemic concentration of the antipsychotic is obtained within 15 minutes of initiation of administration. In certain embodiments, the therapeutic systemic concentration of the antipsychotic is obtained within 1 minute, 2 minutes, 3 minutes, 5 minutes, 10 minutes, 15 minutes, or 30 minutes of initiation of administration when the antipsychotic is prochlorperazine. In certain embodiments, the therapeutic systemic concentration of the antipsychotic is 20 ng/mL or less. In certain embodiments, the therapeutic systemic concentration is 1 ng/mL, 1.5 ng/mL, 2.0 ng/mL, 2.5 ng/mL, 5 ng/mL, 7.5 ng/mL, 10.0 ng/mL, 12.5 ng/mL, or 15 ng/mL of prochlorperazine, within 1 minute, 2 minutes, 3 minutes, 5 minutes, 10 minutes, 15 minutes, or 30 minutes of administration.

In certain embodiments, the methods provide rapid headache relief. In certain embodiments, headache severity is decreased in a patient at a time point 30 minutes or less following initiation of administration of the antipsychotic. In certain embodiments, headache severity is decreased in the patient at a time point 15 minutes or less following initiation of administration of the antipsychotic. In certain embodiments, headache severity is decreased in the patient at a time point 5 minutes or less following initiation of administration of the antipsychotic. In certain embodiments, headache severity is decreased at a time point 5 minutes, 10 minutes, 15 minutes, 20 minutes, 25 minutes, 30 minutes, 35 minutes, 40 minutes, 45 minutes, 50 minutes, 55 minutes, 60 minutes, 75 minutes, 90 minutes, 105 minutes, or 120 minutes following initiation of administration of the antipsychotic. In certain embodiments, headache severity is decreased in the patient at a time point 12 hours or more following initiation of administration of the antipsychotic. In certain embodiments, headache severity is decreased at a time point 2 hours, 4 hours, 8 hours, 12 hours, 16 hours, or 24 hours or more following initiation of administration of the antipsychotic. In certain embodiments, headache severity is decreased in the patient at a time point 30 minutes or less following initiation of administration of the antipsychotic and at a time point 4 hours or more following initiation of administration of the antipsychotic. In certain embodiments, headache severity is decreased at a time point 2 hours or less following initiation of administration of the antipsychotic and at a time point 12 hours or more following initiation of administration of the antipsychotic.

In certain embodiments, headache relief is statistically significant compared to baseline at a time point of about 5 minutes to 120 minutes following initiation of administration of the antipsychotic. In certain embodiments, headache relief is statistically significant compared to baseline at a time point 5 minutes, 10 minutes, 15 minutes, 20 minutes, 25 minutes, 30 minutes, 35 minutes, 40 minutes, 45 minutes, 50 minutes, 55 minutes, 60 minutes, 75 minutes, 90 minutes, 105 minutes, or 120 minutes following initiation of administration of the antipsychotic. In certain embodiments, headache relief is statistically significant compared to baseline at a time point of about 2 hours to 24 hours or more following initiation of administration of the antipsychotic. In certain embodiments, headache relief is statistically significant compared to baseline at a time point 2 hours, 4 hours, 8 hours, 12 hours, 16 hours, or 24 hours or more following initiation of administration of the antipsychotic. In certain embodiments, headache relief is statistically significant compared to baseline at a time point 30 minutes or less following initiation of administration of the antipsychotic and at a time point 4 hours or more following initiation of administration of the antipsychotic. In certain embodiments, headache relief is statistically significant compared to baseline at a time point 2 hours or less following initiation of administration of the antipsychotic and at a time point 12 hours or more following initiation of administration of the antipsychotic.

In certain embodiments, the patient is headache free at a time point 15 minutes or less following initiation of administration of the antipsychotic. In certain embodiments, the patient is headache free at a time point of about 5 minutes to 120 minutes following initiation of administration of the antipsychotic. In certain embodiments, the patient is headache free at a time point 5 minutes, 10 minutes, 15 minutes, 20 minutes, 25 minutes, 30 minutes, 35 minutes, 40 minutes, 45 minutes, 50 minutes, 55 minutes, 60 minutes, 75 minutes, 90 minutes, 105 minutes, or 120 minutes following initiation of administration of the antipsychotic. In certain embodiments, the patient is headache free at a time point of about 2 hours to 24 hours or more following initiation of administration of the antipsychotic. In certain embodiments, the patient is headache free at a time point 2 hours, 4 hours, 8 hours, 12 hours, 16 hours, or 24 hours or more following initiation of administration of the antipsychotic. In certain embodiments, the patient is headache free at a time point 30 minutes or less following initiation of administration of the antipsychotic and at a time point 4 hours or more following initiation of administration of the antipsychotic. In certain embodiments, the patient is headache free at a time point 2 hours or less following initiation of administration of the antipsychotic and at a time point 12 hours or more after initiation of administration of the antipsychotic.

In certain embodiments, the patient self-administers one or more doses of the antipsychotic. In certain embodiments, the patient self-administers a first dose of the antipsychotic, assesses relief after a given interval of time, and, if sufficient headache relief is not obtained, self-administers one or more additional doses of the antipsychotic. In certain embodiments, the first dose is about 0.5 mg to 18 mg of the antipsychotic. In certain embodiments, the first dose is 0.5 mg, 1 mg, 1.5 mg, 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, or 18 mg of the antipsychotic. In certain embodiments, the one or more additional doses are about 1 mg to 18 mg of the antipsychotic. In certain embodiments, the one or more additional doses are 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, or 18 mg of the antipsychotic. In certain embodiments, the given interval of time is the amount of time it takes for the antipsychotic to approximately reach peak plasma concentration. In certain embodiments, the given interval of time is 20 minutes or less. In certain embodiments, the given interval of time is 1 minute, 2 minutes, 5 minutes, 7.5 minutes, 10 minutes, 12.5 minutes, 15 minutes, 20 minutes, 30 minutes, 60 minutes, or 120 minutes. In certain embodiments, the patient self-administers 5 or fewer doses of antipsychotic to decrease the headache. In certain embodiments, the patient is able to essentially titrate to headache relief, thereby reducing side effects such as sedation and akathesia.

In certain embodiments, the antipsychotic is prochlorperazine. In certain embodiments, less than 6 mg of prochlorperazine is administered. In certain embodiments, the administration of the antipsychotic is via inhalation. In certain embodiments, the antipsychotic to be inhaled is a condensation aerosol comprising prochlorperazine.

a. Kit Embodiments

In certain embodiments, kits for the treatment of a headache comprising an antipsychotic and an inhalation delivery device are provided. In certain embodiments, the antipsychotic is selected from acetophenazine, alizapride, amisulpride, amoxapine, amperozide, aripiprazole, benperidol, benzquinamide, bromperidol, buramate, butaclamol, butaperazine, carphenazine, carpipramine, chlorpromazine, chlorprothixene, clocapramine, clomacran, clopenthixol, clospirazine, clothiapine, clozapine, cyamemazine, droperidol, flupenthixol, fluphenazine, fluspirilene, haloperidol, iloperidone, loxapine, melperone, mesoridazine, metofenazate, molindone, perphenazine, pimozide, prochlorperazine, promethazine, olanzapine, penfluridol, pericyazine, pipamerone, piperacetazine, pipotiazine, promazine, remoxipride, risperidone, sertindole, spiperone, sulpiride, thiothixene, thioridazine, trifluoperazine, trifluperidol, ziprasidone, zotepine, and zuclopenthixol. In certain embodiments, the kits comprise a phenothiazine antipsychotic. In certain embodiments, the kits comprise a phenothiazine antipsychotic which is selected from prochlorperazine, trifluorperazine, fluphenazine, promethazine, perphenazine, chlorpromazine, thioridazine, mesoridazine, and acetophenazine. In certain embodiments, the phenothiazine antipsychotic is about 1 to 18 mg prochlorperazine.

In certain embodiments, the kits comprise more than one dose of phenothiazine antipsychotic. In certain embodiments, the kits further comprise instructions for use. In certain embodiments, the kits comprise an inhalation delivery device which produces a condensation aerosol.

### EXAMPLES

### Example 1

### A Toxicokinetic Study of Inhaled Prochlorperazine Condensation Aerosol in the Beagle Dog

This study investigated the systemic absorption of prochlorperazine aerosol delivered by oropharyngeal inhalation in a 5-day repeat dose study in the beagle dog. The research was conducted in Canada at the contract research organization CTBR in compliance with CTBR's Standard Operating Procedures and FDA standard for Good Laboratory Practice (GLP).

Three male and three female beagle dogs were purchased from Covance Research Product, Route 2, Box 113, Cumberland, Va. 23040. The dogs were approximately 7 months to 10 months of age and 6 kg to 12 kg at the onset of treatment. Animals were housed individually in stainless steel cages equipped with a bar-type floor and an automatic watering valve. Each animal was uniquely identified by a permanent tattoo number and/or letter on the ventral aspect of one pinna. Each cage was clearly labeled with a color-coded cage card indicating project, group, animal number, tattoo number, and sex. The environmental conditions of the animal room were standardized. The temperature was maintained at 20° C.±3° C., the humidity was kept at 50%±20% humidity, and the light cycle was 12 hours light and 12 hours dark, except during designated procedures. An acclimation period of approximately 3 weeks was allowed between animal receipt and the start of treatment in order to accustom the animals to the laboratory environment.

All animals had access to a standard certified pelleted commercial dog food (400 g—PMI Certified Dog Chow 5007: PMI Nutrition International Inc.) except during designated procedures. Maximum allowable concentrations of contaminants in the diet (e.g., heavy metals, aflatoxin, organophosphate, chlorinated hydrocarbons, and PCBs) were controlled. Municipal tap water which had been softened, purified by reverse osmosis and exposed to ultraviolet light was freely available, except during designated procedures.

Animals were treated with the antipsychotic aerosol using an oropharyngeal facemask fitted with inlet and outlet tubes. This mask included a plastic cylinder and was fitted over the dog's muzzle in such a way that the nose was inside the cylinder and the animal was mouth breathing through a short tube. The test antipsychotic was generated by vaporizing prochlorperazine by heating to roughly 400° C. an approximately 8 micron thick film of prochlorperazine which had been formed on stainless steel foil by dip coating the foil into a solution of prochlorperazine dissolved in organic solvent. The resulting aerosol formed by the condensation of the vaporized prochlorperazine was introduced into a mixing chamber via pre-dried compressed air. The mixing chamber was operated under slight positive pressure maintained by a gate valve located in the exhaust line. A vacuum pump was used to exhaust the inhalation chamber at the required flow rate and draw the contaminated air (excess aerosol and expired air) through a purifying system including a 5 μm coarse filter before expelling the air from the building. The resulting atmosphere was carried to the dog mask via a delivery tube. During treatment, animals were placed in a restraint sling.

The homogeneity of chamber atmosphere concentration was determined by collecting filter samples in duplicate for gravimetric and HPLC analysis of prochlorperazine content from 2 equidistantly spaced dog breathing ports located about the circumference of the mixing chamber. Additional samples were also collected from a reference port to assess total prochlorperazine distribution variation within the chamber and also within-port variation in prochlorperazine distribution. The results obtained from this analysis demonstrated uniform aerosol distribution.

Analysis of the aerosol particle size distribution was conducted using a Cascade Impactor. The method included classification into a series of size ranges followed by gravimetric and HPLC analyses. The mass median aerodynamic diameter and its geometric standard deviation (MMAD±GSD) were calculated from the gravimetric and HPLC data using a computer program based on the Andersen Operating Manual TR#76-900016, and was found to be about 1.5 μm ±2 μm.

The attained dose of active ingredient (prochlorperazine) (mg/kg/day) was determined as follows, with numerical values in the table below being the mean value of the parameter among all tested dogs (N=3 males and N=3 females):

| | |
|---|---|
| Achieved Dose of active =Ingredient (mg/kg/day) | $\frac{RMV \times Active Concentration \times T \times D}{BW} = 12.3 mg / kg / d$ |
| Where RMV (L/min) = | respiratory minute volume* = 6.27 L/min |
| Active Concentration = | chamber concentration of active ingredient determined |
| (mg/L) | by chemical analysis = 3.0 mg/L |
| T (min) = | treatment time = 10 minutes |
| D = | total aerosol deposition fraction, according to the |
| | particle size⁽¹⁾ = 0.50 |
| BW (kg) = | weight = 7.7 kg |

*Measured using the Buxco Electronics LS-20 system for each animal twice prior to first prochlorperazine treatment.
⁽¹⁾As described in Witschi & Nettesheim, Mechanisms in Respiratory Toxicology, Vol. 1: 54-56, CRC Press, Inc. 1982

Dogs were treated with aerosol as above for 10 minutes daily for 5 consecutive days. On the first and last day (days 1 and 5), plasma samples were collected for toxicokinetic analysis 2 minutes after the initiation of inhalation, immediately after dosing, and 20 minutes, 1.5 hours, 6 hours, and 24 hours post dosing (i.e., 10 minutes, 30 minutes, 100 minutes, 370 minutes and 1450 minutes after initiation of inhalation). On day 5, a sample was also collected immediately prior to dosing. Samples were stored at −80° C. until prochlorperazine plasma concentration analysis was performed.

Prochlorperazine plasma concentration in the samples was measured by liquid chromatography-mass spectrometry/mass spectrometry (“LC-MS/MS”) using a validated analytical method. A standard curve was used covering the nominal concentration range of 2 ng/mL to 400 ng/mL. To each study sample (dog plasma containing EDTA) an aliquot of internal standard (tritiated-prochlorperazine) was added. The samples were then mixed with sodium bicarbonate solution and acetonitrile and analyzed (5 μL injection volume). Chromatography equipment was Agilent 100 series HPLC with UpChurch A-355 Peek precolumn filter and A-707 Peek Frit and a Phenomenex Synergi Hydro-RP (4 μm bead, 80 angstrom pore size) main column of 50 mm length and 3 mm internal diameter. Chromatography conditions were temperature 45° C., mobile phase A (“A”) of 10 mM ammonium acetate buffer in water (pH 3) and mobile phase B (“B”) of 0.05% formic acid in acetonitrile with starting conditions of 30% B for the first 0.5 minutes, then ramping over 2.5 minutes to 90% B (maintained for 2 minutes) and than ramping over 0.2 minutes to 30% B (maintained for 0.8 minutes) for a total running time of 6 minutes at a total flow rate of 0.5 mL/minute. MS/MS equipment was MDS Sciex API 3000 system with electrospray positive ionization and multiple reaction monitoring scanning. Under the above conditions, prochlorperazine (MW 374) eluted at 3.3 minutes as did the internal standard (MW 377). The coefficient of variance of the analytical method was determined using calibration standards of 6 ng/mL, 60 ng/mL, and 300 ng/mL. The coefficient of variance and was found to be<5%.

Results from the dogs (mean concentrations of prochlorperazine in ng/mL±standard deviation across the 3 dogs of the same gender) were as follows:

| | Treat- | 2 min. | | | | | |
|---|---|---|---|---|---|---|---|
| Dog | ment | into | 0 min. | 20 min. | 1.5 hrs | 6 hrs | 24 hrs |
| Gender | Day | dose | post | post | post | post | post |
| Male | 1 | 860 ± | 1660 ± | 974 ± | 349 ± | 107 ± | 12 ± 3 |
| (N = 3) | | 422 | 19 | 253 | 80 | 60 | |
| Female | 1 | 841 ± | 2208 ± | 1036 ± | 499 ± | 175 ± | 14 ± 9 |
| (N = 3) | | 204 | 633 | 229 | 70 | 54 | |
| Male | 5 | 568 ± | 1533 ± | 1038 ± | 664 ± | 272 ± | 96 ± 35 |
| (N = 3) | | 432 | 353 | 52 | 88 | 72 | |
| Female | 5 | 829 ± | 1877 ± | 1272 ± | 593 ± | 340 ± | 86 + 67 |
| (N = 3) | | 319 | 536 | 426 | 130 | 110 | |

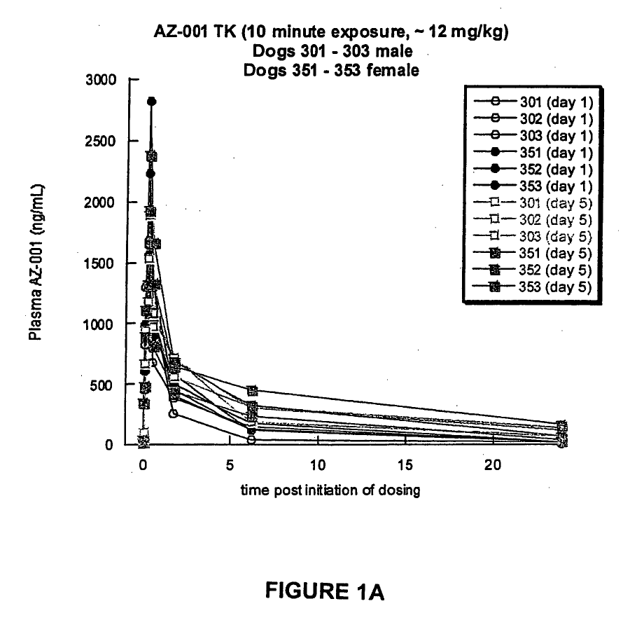

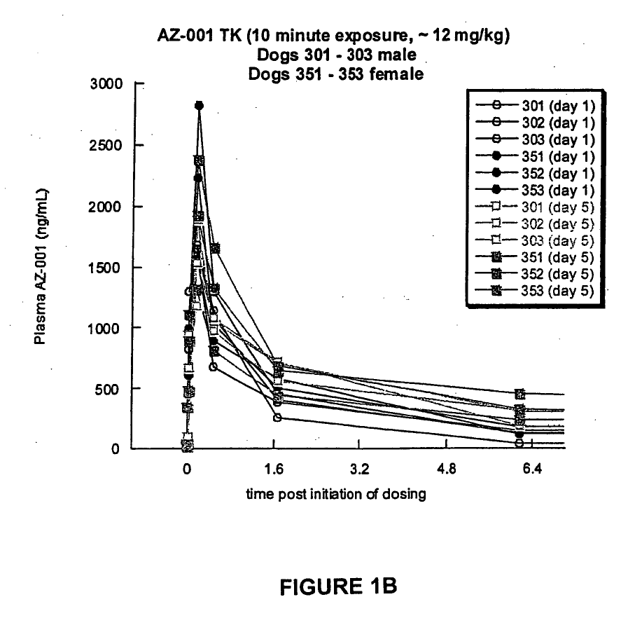

Individual animal results are shown in FIG. 1A (from prior to treatment to 24 hours post treatment) and FIG. 1B (identical data to those shown in FIG. 1A but focusing on the time from initiation of treatment to 6.4 hours post treatment).

Pre-dose concentrations of prochlorperazine on Day 5 were: male 19 ng/mL, 30 ng/mL, and 10 ng/mL for the three dogs and female 40 ng/mL, 23 ng/mL, and 341 ng/mL for the three dogs.

In this study, prochlorperazine plasma concentration rose very rapidly after aerosol administration, with the peak plasma concentration obtained approximately at the end of prochlorperazine inhalation. The rate of rise in prochlorperazine plasma concentration was found to be >4 ng/mL/minute, >8 ng/mL/minute, and even >20 ng/mL/minute. Therapeutic plasma levels of approximately at least 0.5 ng/mL, 1 ng/mL, 2 ng/mL, 4 ng/mL, 8 ng/mL, and even 15 ng/mL were obtained within 10 minutes of initiation of administration of prochlorperazine, and even within 2 minutes of initiation of administration of prochlorperazine.

### Example 2

### A 17-Day Repeat Dose Toxicity Study of Inhaled Prochlorperazine Condensation Aerosol in the Beagle Dog

This study investigated the potential toxicity of three different doses of prochlorperazine aerosol delivered by oropharyngeal inhalation in a 17-day repeat dose study in the beagle dog.

This research was conducted at the same location as in Example 1, and using the same Standard Operating Procedures and Good Laboratory Practice requirements as in Example 1. The beagle dogs were purchased from the same vendor and housed and identified as described in Example 1. The animal room environmental conditions were as described in Example 1. As in Example 1, an acclimation period of approximately 3 weeks was allowed between animal receipt and the start of treatment in order to accustom the animals to the laboratory environment.

Before initiation of administration of the antipsychotic, all animals were weighed and assigned to treatment groups using a randomization procedure. Randomization was by stratification using body weight as the parameter. Males and females were randomized separately. Final animal allocation was checked to ensure that littermates were homogeneously distributed across all groups. Animals were assigned into the following groups: repeat dose prochlorperazine 2 mg/kg (3 males and 3 females), repeat dose prochlorperazine 0.5 mg/kg (3 males and 3 females), repeat dose prochlorperazine 0.125 mg/kg (3 males and 3 males) and vehicle control repeat dose (3 males and 3 females).

The oropharyngeal inhalation apparatus and setup were identical to those described in Example 1. As in Example 1, animals were placed in a restraint sling during treatment.

The vehicle control group was exposed to predried compressed air passed through the antipsychotic-heating apparatus with the apparatus loaded with clean stainless steel foil instead of prochlorperazine-coated foil. Except for the absence of prochlorperazine, exposure in the vehicle control group was identical to that in the 2 mg/kg repeat dose group with regard to the air being passed through the operating and heating apparatus, the dogs breathing only through the dog masks, and the dogs being restrained and handled in the same manner.

To ensure that the doses were correct, atmosphere characterization of the test article aerosol was performed. The operational conditions of the exposure system required to establish each target aerosol concentration were determined gravimetrically and by HPLC analysis of prochlorperazine content from open-face glass fiber filter samples collected at a representative animal exposure mask.

The homogeneity of chamber atmosphere concentration was also determined at the 0.125 mg/kg and 2 mg/kg dose levels for prochlorperazine. This comprised collecting filter samples in duplicate for gravimetric and HPLC analysis from two equidistantly spaced dog breathing ports located about the circumference of the mixing chamber. Additional samples were also collected from a reference port to assess total prochlorperazine distribution variation within the chamber and also within-port variation in prochlorperazine distribution. The results obtained from this analysis demonstrated uniform aerosol distribution.

Analysis of the aerosol particle size distribution for each prochlorperazine dose was conducted using a Cascade Impactor. The method included classification into a series of size ranges followed by gravimetric and HPLC analysis. The mass median aerodynamic diameter and its geometric standard deviation (MMAD±GSD) were calculated from the gravimetric data using a computer program based on the Andersen Operating Manual TR#76-900016. Typical mass median aerodynamic diameter and GSD measured during the study were 1.4 μm±2.2.

Actual mask output concentrations of aerosol were measured at least once during each exposure day from a sampling port in the animal breathing zone using a gravimetric and/or HPLC method.

The achieved dose of active ingredient (prochlorperazine) (mg/kg/day) for each treatment level was determined as follows:

| | |
|---|---|
| Achieved Dose of active =Ingredient (mg/kg/day) | $\frac{RMV \times Active Concentration \times T \times D}{BW}$ |
| Where RMV (L/min) = | respiratory minute volume* |
| Active = | chamber concentration of active ingredient determined |
| Concentration (mg/L) | by chemical analysis. |
| T (min) = | treatment time |
| D = | total aerosol deposition fraction, according to the |
| | particle size⁽¹⁾ |
| BW (kg) = | mean body weight per sex per group from the regular |
| | body weight occasions during treatment. |

*Measured using the Buxco Electronics LS-20 system for each animal twice prior to first prochlorperazine treatment.
⁽¹⁾As described in Witschi & Nettesheim, Mechanisms in Respiratory Toxicology, Vol. 1: 54-56, CRC Press, Inc. 1982

Dogs were treated with the prochlorperazine aerosol using the above approach to deliver the drug aerosol and compute the delivered dose. Exposure duration was adjusted to ensure achieving the target doses of 0.125 mg/kg, 0.5 mg/kg and 2 mg/kg, with the required dosing durations 13 minutes, 15 minutes, and 7 minutes respectively, with higher chamber aerosol concentrations used for the higher doses (thus, only 7 minutes delivered the largest total dose of 2 mg/kg, whereas longer dosing was used to deliver the lower doses). Dosing occurred on study days 1, 5, 9, 13, and 17, with no drug given on the other days.

Animals were observed for signs of drug effects during the treatment period. At the 2 mg/kg dose level, the dogs were noted to have decreased activity and weakness following dosing. In addition, occasional coughing occurred. The classic signs of bronchoconstriction (wheezing, prolonged expiratory phase, and difficulty with respiration) were not found at any dose level.

Animals were necropsied on completion of the treatment period by exsanguination by incision of the axillary or femoral arteries following anesthesia by intravenous injection of sodium pentobarbital. A sedative, Ketamine HCl for Injection, U.S.P. and Xylazine, was administered by intramuscular injection before animals were transported from the animal room to the necropsy area. In order to avoid autolytic change, a complete gross pathology examination of the carcass was conducted immediately on all animals which were euthanized. Food was withheld from all animals overnight before scheduled necropsy. No treatment related findings were detected during necropsy for any of the animals. Histopathological examination of any gross lesions was conducted. Again, no treatment related findings were observed. In addition, histopathological examination of the larynx, trachea, mainstem bronchi, lungs including bronchi, and nasal cavities was conducted. No treatment related abnormalities were observed.

### Example 3

### Intravenous Dose-Ranging Efficacy Study of Prochlorperazine for Migraine

The following study showed that prochlorperazine administered intravenously to patients in doses less than 10 mg provided relief for moderate to severe migraine or tension-type headache. Certain other studies had previously been performed to evaluate the efficacy of intravenous prochlorperazine in headache treatment, but only at doses of 10 mg or above by the intravenous or intramuscular routes of administration.

Potential participants in the study were screened prior to enrollment in the study (hereinafter “screening”). The general health of the potential participants was assessed by medical history, physical examination, 12-lead electrocardiograms (“EGCs”), blood chemistry profile, hematology, and urinalysis. Vital signs were assessed once after the potential participant had been in a sitting position for at least 5 minutes and again after the potential participant had been in the standing position for at least 3 minutes.

Blood samples were collected according to standard medical guidelines. Blood and urine samples were shipped according to instructions from the local laboratory. Blood was collected in non-anticoagulated, evacuated, venous blood collection tubes (e.g., Vacutainer™), and the serum separated according to standard procedures. Quantitative analyses were performed for the following analytes: alkaline phosphatase, albumin, bicarbonate, calcium, total cholesterol, chloride, creatine kinase (CK), creatinine, glucose, inorganic phosphorus, potassium, alanine aminotransferase, aspartate aminotransferase, sodium, total bilirubin, total protein, urea, and uric acid. Blood was also collected in anticoagulant-containing, evacuated, venous blood collection tubes (e.g., Vacutainer™) for haematology testing according to standard procedures. Quantitative analyses were performed for the following analytes: hemoglobin, hematocrit, red blood cell count with indices, white blood cell count, white blood cell differential, and platelet count.

A mid-stream urine sample was collected in a clean container. Qualitative analyses were performed for the following analytes: specific gravity, pH, acetone, albumin, glucose, urobilinogen, protein, blood, and bilirubin.

Twelve-lead ECGs were performed at all study visits according to standard procedures, and were interpreted by a qualified physician. All medications (prescription and non-prescription, herbal medications or investigational drugs) taken by the subjects during the 28 days prior to the screening baseline period were documented. All such medications were reviewed and evaluated by the Principal Investigator or designate to determine if they affected the potential participant's eligibility to participate in the study.

Potential participants were also screened for various risk factors. Potential participants with indications of drug or alcohol dependence within the prior 12 months (excepting tobacco dependence) were excluded. Female potential participants at risk of becoming pregnant were not enrolled unless they had a negative pregnancy test both at the time of screening and upon admission to the clinic for the administration of prochlorperazine. Both male and female participants agreed to use a medically acceptable and effective birth control method throughout the study. Participants understood English sufficiently well to give their informed consent, and further agreed to adhere to the study visit schedule and to complete the protocol-specified assessments.

Potential participants with a known history of allergy, intolerance, or history of contraindications to the use of phenothiazines, anticholinergics and related drugs were not eligible for inclusion in the study. Potential participants taking other headache medications within 24 hours prior to admission to the clinic for study treatment were also excluded. Potential participants taking lithium or monoamine oxidase inhibitors were not included in the study. Potential participants having received an investigational drug within 3 months prior to screening were similarly excluded. Potential participants with a known history of pheochromocytoma, seizure disorder, Parkinson's disease, Restless Leg Syndrome, unstable angina, syncope, coronary artery disease, myocardial infarction, congestive heart failure, stroke, transient ischemic attack, uncontrolled hypertension, or clinically significant ECG abnormality were excluded as well.

The study was a double-blind, randomized, placebo-controlled, dose-ranging, single center trial of intravenous prochlorperazine (Stemetil® Injectable) in patients with moderate to severe migraine or tension-type headaches. Participating in the study were 80 male and female subjects (22 males and 58 females), ranging in age from 19.4 to 59.1 years). All subjects had a history of moderate to severe headache by self-report (migraine with or without aura, or tension-type headache) with an average frequency of 1-6 attacks per month during the prior three months. Of these subjects, 51 had moderate to severe migraine headache and 29 had moderate to severe tension headache, as assessed by a physician upon presentation to the clinic for administration of prochlorperazine. There were no apparent differences between the two headache groups or across treatment groups in terms of age, gender, or weight.

Upon admission to the clinic for administration of prochlorperazine, re-confirmation of continued eligibility of the study participants for the study was made. Vital signs of the participants were measured after the subject had been in the sitting position for at least 5 minutes. Orthostatic measurements of systolic and diastolic blood pressure were also taken. Supine blood pressure was taken after the subject had been in the supine position for 5 minutes. The subject then stood and the measurement was repeated at 1 minute and 3 minutes after standing. Upon re-confirmation of eligibility, pre-treatment headache severity as determined by the patient's self perception of the headache was recorded on a standard 4-point categorical scale where 0 indicated the absence of headache, 1 indicated mild headache, 2 indicated moderate headache, and 3 indicated severe headache. Pre-treatment severity of nausea, sedation, and akathisia was similarly recorded on a 4-point scale. The presence or absence of photophobia and phonophobia was recorded on a 2-point scale (does the light make your headache worse? 0—No, 1—Yes; does noise make your headache worse? 0—No, 1—Yes).

Fifteen minutes after completing the above assessment, study participants were administered a single dose of intravenous prochlorperazine (1.25 mg, 2.5 mg, 5 mg, or 10 mg) or placebo (saline) in a standard volume of 5 mL made up with normal saline. Administration was over 2±1 minutes by infusion pump. Neither the study participant, nor the study center staff conducting the drug treatment sessions were aware of which treatment was being administered.

Response to treatment was determined by assessing patients at 15, 30, 60, and 120 minutes following drug administration using the above scales for severity of headache, nausea, sedation, akathisia, and the presence or absence of photophobia, and phonophobia. Following discharge form the clinic, participants were asked the same questions, and recorded their responses in a diary at 4, 8, and 24 hours post-treatment.

Each subject also rated the amount of relief of headache pain experienced at 15, 30, 60, and 120 minutes following prochlorperazine administration. Following discharge from the clinic, these measures were also assessed and recorded by the subject in a diary at 4, 8, and 24 hours post-treatment. The subject rated the amount of pain relief provided by the study treatment using a categorical 5-point scale (1—no pain relief, 2—some pain relief, 3—moderate pain relief, 4—much pain relief, and 5—complete pain relief).

Each subject also assessed the efficacy of study treatment at 120 minutes and 24 hours post-treatment in the subject diary. Subjects rated their satisfaction with the pain relief provided by the study treatment using a categorical 5-point scale (1—very poor, 2—poor, 3—no opinion, 4—good, and 5—very good).

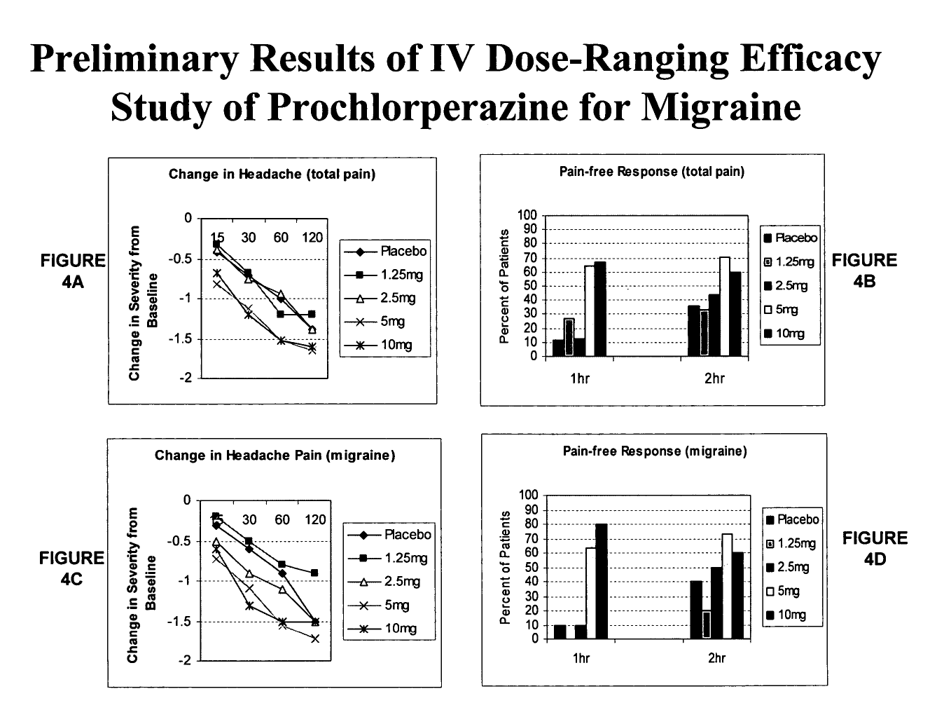

Migraine headache severity was most effectively reduced at 60 minutes after initiation of administration of prochlorperazine by the 5 mg dosage (mean decrease in severity: −1.55), which was even more effective than the 10 mg dosage (mean decrease in severity: −1.50. The 2.5 mg dosage (mean decrease in severity −1.18) was also more effective than placebo (mean decrease in severity −1.10). See FIGS. 4C and 4D. Tension headache severity was most effectively reduced at 60 minutes after initiation of administration of prochlorperazine by the 1.25 mg dosage (mean decrease in severity: −2.00), the 5 mg dosage (mean decrease in severity: −1.50), and the 10 mg dosage (mean decrease in severity: −1.60). For both types of headaches taken together, the 5 mg dose (mean decrease in severity: −1.53) and the 10 mg dose (mean decrease in severity: −1.53) were most effective, with the 5 mg dosage just as effective as the 10 mg dosage. See FIGS. 4A and 4B.

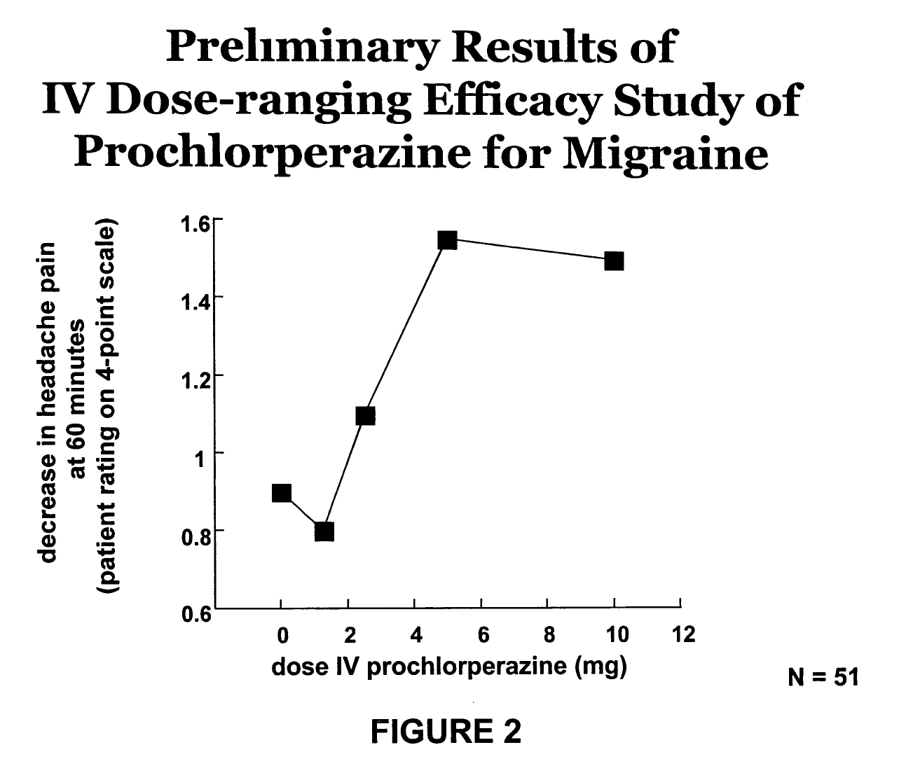

At 15 and 30 minutes post administration of prochlorperazine, the 5 mg and 10 mg doses caused the largest decrease in headache severity, with 5 mg again approximately as effective or more effective than 10 mg. See FIG. 4C. See also FIG. 2.

A remarkable advantage of even low doses of prochlorperazine compared to placebo was noted based on the percentage of patients pain free (as defined by an absence of headache pain on the self-reported headache severity scale) at 1 and 2 hours post treatment initiation. In particular, at 1 hour post treatment only 11.8% of placebo-treated patients were pain free, whereas 26.7% of patients receiving 1.25 mg of prochlorperazine were pain free. At the 5 mg dose, the percentage of pain free patients increased to 64.7%, similar to the 66.7% in the 10 mg dose group. At 2 hours post treatment, only 35.3% of placebo-treated patients were pain free, compared to 43.8% in the 2.5 mg dose group, 70.6% in the 5 mg dose group, and 60% in the 10 mg dose group.

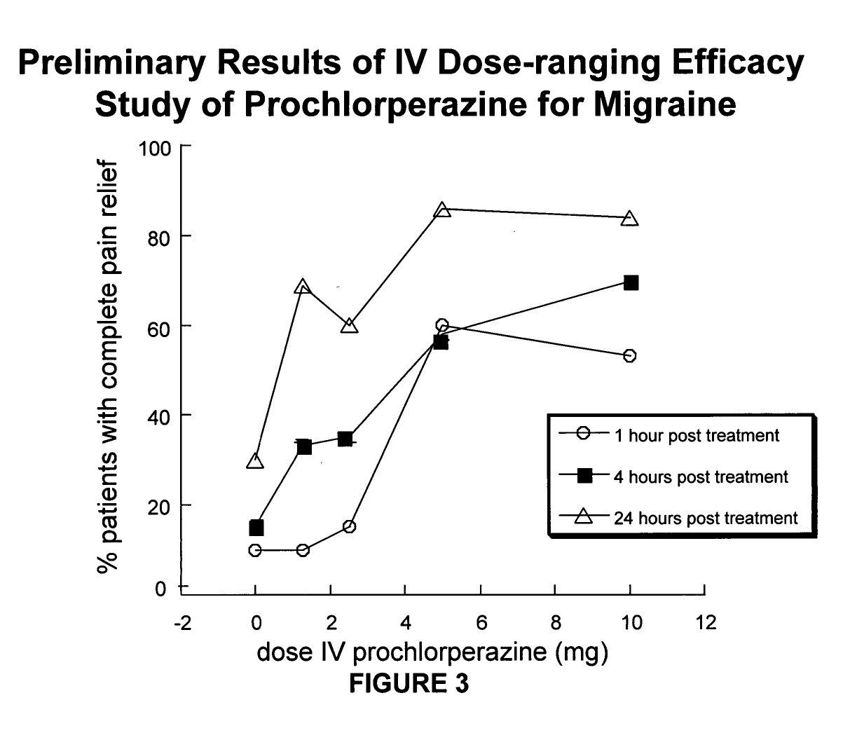

Similar data relating to patients pain free, in this case measured as complete pain relief on the pain relief scale, is shown in FIG. 3. Note that at 1 hour, there is only a small benefit of prochlorperazine doses <2.5 mg on pain relief by this measure (in contrast to some other measures), but that 5 mg is exceptionally effective by this measure as it is by virtually all measures. By 4 hours post treatment, remarkably, doses as low as 1.25 mg show meaningfully greater efficacy than placebo (0 mg). Even more remarkably at 24 hours, even the lowest tested dose of 1.25 is very effective, whereas placebo is not. Outcome at 24 hours is critical in migraine, because the natural history of migraine involves a headache lasting often up to 72 hours.

Echoing the results shown in FIG. 3, but in this case specific to migraine sufferers, 24 hours after initiation of administration of prochlorperazine, 84-88% of those subjects receiving 1.25 mg, 5 mg or 10 mg doses were free of pain, compared to less than half of subjects with migraines who received placebo, providing strong evidence for the effectiveness of prochlorperazine in the low dose of 1.25 mg in the treatment of migraine headache With tension headache sufferers, 80% of participants who received the 2.5 mg dose were free of pain at 24 hours, as were >80% who received 5 mg or 10 mg of prochlorperazine, whereas a minority of patients receiving placebo were pain free, providing strong evidence for the effectiveness of prochlorperazine in the low dose of 2.5 mg in the treatment of tension headache.

Ninety percent or more of participants receiving the 5 mg or 10 mg doses had at least some pain relief 15 minutes after initiation of administration of prochlorperazine, and there were no subjects in these treatment groups that did not obtain at least some pain relief. Pain relief was not obtained as rapidly in participants receiving the 0 mg, 1.25 mg, and 2.5 mg doses in comparison to those receiving the 5 mg and 10 mg doses. The largest proportion of patients with migraines to report being free of pain was in the 5 mg and 10 mg dosage groups at both 2 hours and 24 hours. Participants with tension headaches more frequently reported being free of pain at 2 hours and 24 hours after receiving the 1.25 mg or 5 mg doses. The 10 mg dose also resulted in a large proportion of participants with tension headaches to report being free of pain at 24 hours.

The subjects' global evaluation of their treatment at 2 and 24 hours after initiation of administration of prochlorperazine was in favor of the 5 mg and 10 mg dosages, with the 2.5 mg dose also preferred to placebo, at least in patients with migraine headache, further confirming the clinical value of these low ≦5 mg) prochlorperazine doses.

Fifty-three of the 80 subjects experienced dose related adverse events. Ninety-four percent of all adverse events were mild to moderate in intensity, with only 6% judged as severe. The most frequently observed adverse events were drowsiness and restlessness, accounting for 52% and 18% of the adverse events, respectively. Adverse effects were reported more frequently in the 5 mg and 10 mg dosage groups as compared to other treatment groups. The classical prochlorperazine side effect of akathisia was more common in the 10 mg dose group than other groups. These adverse event data further support the above efficacy data which point to the remarkable clinical value of using doses <10 mg.

Rescue medications for headache were taken by only 9 of 80 subjects (11%). Of these subjects, 3 received the 2.5 mg dose, 2 received placebo, 3 received the 1.25 mg dose, 1 received the 5 mg dose, and none received the 10 mg dose. This showed a trend of less use of medication for headache in 5 mg and 10 mg groups as compared to the other groups, although the numbers were small. There was no apparent difference in the use of medication for headaches between headache types. Medications for headache included Advil, Excedrin, ibuprofen, propranolol, Tylenol, Tylenol #2, and Tylenol #3.

Overall, the low prochlorperazine doses tested of 1.25 mg, 2.5 mg, and 5 mg all showed substantial clinical efficacy at certain time points and in certain clinical endpoints in both migraine and tension headache patients. The 5 mg dose was equally effective as the 10 mg dose in this study.

The above results were based on 15 to 17 patients per treatment group. To determine accurately the statistical significance of the clinical benefits described above at particular dose levels, a larger sample size than that studied above would be required, although the above data would be sufficient for a statistician skilled in the art to establish, by constructing appropriate composite measures, the statistical significance of the overall effectiveness of the low prochlorperazine doses of 1.25 mg to 5 mg. To determine statistical significance in a dose by dose manner, however, in addition to defining end-points prior to the study to avoid statistical problems with multiple comparisons, it would be advantageous to have at least 30 patients per group, with markedly greater chances of observing statistical significance with 50, 75, 100, 150, 200, or 300 patients per group. Such numbers of patients are commonly enrolled per group in pivotal clinical trials of headache medications.

### Example 4

### Certain General Methods

In Method 1, an antipsychotic-coated aluminum foil substrate is prepared a substrate of aluminum foil (10 cm×5.5 cm; 0.0005 inches thick) was precleaned with acetone. A solution of antipsychotic in a minimal amount of solvent was coated onto the foil substrate to cover an area of approximately 7-8 cm×2.5 cm. The solvent was allowed to evaporate. The coated foil was wrapped around a 300 watt halogen tube (Feit Electric Company, Pico Rivera, Calif.), which was inserted into a glass tube sealed at one end with a rubber stopper. Sixty volts of alternating current (driven by line power controlled by a Variac) were run through the bulb for 5-15 seconds, or in some studies 90 volts for 3.5-6 seconds, to generate a thermal vapor (including aerosol) which was collected on the glass tube walls. In some studies, the system was flushed through with argon prior to volatilization. The material collected on the glass tube walls was recovered and the following determinations were made: (1) the amount emitted, (2) the percent emitted, and (3) the purity of the aerosol by reverse-phase HPLC analysis with detection by absorption of 225 nm light. The initial antipsychotic mass was found by weighing the aluminum foil substrate prior to and after antipsychotic coating. The thickness of the antipsychotic film was obtained by: film thickness (cm)=antipsychotic mass (g)/[antipsychotic density (g/cm³)×substrate area (cm²).

In Method 2, an antipsychotic-coated stainless steel cylindrical substrate is prepared. A hollow stainless steel cylinder with thin walls, e.g., 0.12 mm wall thickness, a diameter of 13 mm, and a length of 34 mm was cleaned in dichloromethane, methanol, and acetone, then dried, and fired at least once to remove any residual volatile material and to thermally passivate the stainless steel surface. The substrate was then dip-coated with an antipsychotic coating solution (prepared as disclosed below in Method 5). The dip-coating was done using a computerized dip-coating machine to produce a thin layer of antipsychotic on the outside of the substrate surface. The substrate was lowered into the drug solution and then removed from the solvent at a rate of 5-25 cm/sec. (To coat larger amounts of material on the substrate, the substrate was removed more rapidly from the solvent or the solution used was more concentrated.) The substrate was then allowed to dry for 30 minutes inside a fume hood. If either dimethylformamide (DMF) or a water mixture was used as a dip-coating solvent, the substrate was vacuum dried inside a dessicator for a minimum of one hour. In these studies, the antipsychotic-coated portion of the cylinder generally has a surface area of 8 cm². By assuming a unit density for the antipsychotic, the initial antipsychotic coating thickness was calculated. The amount of antipsychotic coated onto the substrates was determined by extracting the coating with methanol or acetonitrile and analyzing the extracted materials with quantitative HPLC methods to determine the mass of drug coated onto the substrate.

In Method 3, an antipsychotic-coated aluminum foil substrate is prepared. A substrate of aluminum foil (3.5 cm×7 cm; 0.0005 inches thick) was precleaned with acetone. A solution of antipsychotic in a minimal amount of solvent was coated onto the foil substrate. The solvent was allowed to evaporate. The coated foil was wrapped around a 300 watt halogen tube (Feit Electric Company, Pico Rivera, Calif.), which was inserted into a T-shaped glass tube sealed at two ends with Parafilm®. The Parafilm® was punctured with ten to fifteen needles for air flow. The third opening was connected to a 1 liter, 3-neck glass flask. The glass flask was further connected to a piston capable of drawing 1.1 liters of air through the flask. Ninety volts of alternating current (driven by line power controlled by a Variac) was run through the bulb for 6-7 seconds to generate a thermal vapor (including aerosol) which was drawn into the 1 liter flask. The aerosol was allowed to sediment onto the walls of the 1 liter flask for 30 minutes. The material collected on the flask walls was recovered and the following determinations were made by reverse-phase HPLC with detection by absorption at 225 nm: (1) the amount emitted, (2) the percent emitted, and (3) the purity of the aerosol. Additionally, any material remaining on the substrate was collected and quantified.

In Method 4, an antipsychotic-coated stainless steel foil substrate is prepared. Strips of clean 304 stainless steel foil (0.0125 cm thick, Thin Metal Sales) having dimensions 1.3 cm by 7.0 cm were dip-coated with an antipsychotic solution. The foil was then partially dipped three times into solvent to rinse antipsychotic off of the last 2-3 cm of the dipped end of the foil. Alternatively, the antipsychotic coating from this area was carefully scraped off with a razor blade. The final coated area was between 2.0-2.5 cm by 1.3 cm on both sides of the foil, for a total area of between 5.2-6.5 cm². Several prepared foils were extracted with methanol or acetonitrile as standards. The amount of antipsychotic was determined by quantitative HPLC analysis. Using the known antipsychotic-coated surface area, the thickness was then obtained by: film thickness (cm)=antipsychotic mass (g)/[antipsychotic density (g/cm³)×substrate area (cm²)]. If the antipsychotic density is not known, a value of 1 g/cm³ is assumed. The film thickness in microns is obtained by multiplying the film thickness in cm by 10,000.

After drying, the antipsychotic-coated foil was placed into a volatilization chamber constructed of a Delrin® block (the airway) and brass bars, which served as electrodes. The dimensions of the airway were 1.3 cm high by 2.6 cm wide by 8.9 cm long. The antipsychotic-coated foil was placed into the volatilization chamber such that the antipsychotic-coated section was between the two sets of electrodes. After securing the top of the volatilization chamber, the electrodes were connected to a 1 Farad capacity (Phoenix Gold). The back of the volatilization chamber was connected to a two micron Teflon filter (Savillex) and filter housing, which were in turn connected to the house vacuum. Sufficient airflow was initiated (about 30 L/min=1.5 m/sec), at which point the capacitor was charged with a power supply, between 14 volts and 17 volts. The circuit was closed with a switch, causing the antipsychotic-coated foil to resistively heat to temperatures of about 280-430° C. (as measured with an infrared camera (FLIR Thermacam SC3000)), in about 200 milliseconds. (For comparison purposes, see FIG. 4A, thermocouple measurement in still air.) After the antipsychotic had vaporized, airflow was stopped and the Teflon® filter was extracted with acetonitrile. Antipsychotic extracted from the filter was analyzed by HPLC UV absorbance generally at 225 nm using a gradient method aimed at detection of impurities to determine percent purity. Also, the extracted antipsychotic was quantified to determine a percent yield, based on the mass of antipsychotic initially coated onto the substrate. A percent recovery was determined by quantifying any antipsychotic remaining on the substrate and chamber walls, adding this to the quantity of antipsychotic recovered in the filter and comparing it to the mass of antipsychotic initially coated onto the substrate.

Method 5 describes the preparation of an antipsychotic-coating solution. Antipsychotic was dissolved in an appropriate solvent. Common solvent choices included methanol, dichloromethane, methyl ethyl ketone, diethyl ether, 3:1 chloroform: methanol mixture, 1:1 dichloromethane: methyl ethyl ketone mixture, dimethylformamide, and dionized water. Sonication and/or heat were used as necessary to dissolve the compound. The resulting antipsychotic concentration was about 50 mg/mL to 200 mg/mL.

### Example 5

### Chlorpromazine

Chlorpromazine (MW 319, melting point <25° C., oral dose 300 mg), an antipsychotic, was coated on an aluminum foil substrate (20 cm 2) according to Method 1. See Example 4.

9.60 mg of chlorpromazine was applied to the substrate, for a calculated thickness of the chlorpromazine film of 4.8 μm. The substrate was heated as described in Method 1 at 90 volts for 5 seconds. The purity of the chlorpromazine-aerosol particles was determined to be 96.5%. 8.6 mg was recovered from the glass tube walls after vaporization, for a percent yield of 89.6%.

### Example 6

### Clozapine

Clozapine (MW 327, melting point 184° C., oral dose 150 mg), an antipsychotic, was coated on an aluminum foil substrate (20 cm²) according to Method 1. See Example 4. 14.30 mg of clozapine was applied to the substrate, for a calculated thickness of the clozapine film of 7.2 μm. The substrate was heated as described in Method 1 at 90 volts for 5 seconds. The purity of the clozapine-aerosol particles was determined to be 99.1%. 2.7 mg was recovered from the glass tube walls after vaporization, for a percent yield of 18.9%.

Another substrate containing clozapine coated (2.50 mg clozapine) to a film thickness of 1.3 μm was prepared by the same method and heated as described in Method 1 under an argon atmosphere at 90 volts for 3.5 seconds. The purity of the clozapine-aerosol particles was determined to be 99.5%. 1.57 mg was recovered from the glass tube walls after vaporization, for a percent yield of 62.8%.

### Example 7

### Haloperidol

Haloperidol (MW 376, melting point 149° C., oral dose 2 mg), an antipsychotic, was coated on an aluminum foil substrate (20 cm²) according to Method 1. See Example 4. 2.20 mg of Haloperidol was applied to the substrate, for a calculated thickness of the haloperidol film of 1.1 μm. The substrate was heated as described in Method 1 at 108 volts for 2.25 seconds. The purity of the haloperidol-aerosol particles was determined to be 99.8%. 0.6 mg was recovered from the glass tube walls after vaporization, for a percent yield of 27.3%.

Haloperidol was further coated on an aluminum foil substrate according to Method 1. See Example 4. When 2.1 mg of haloperidol was heated as described in Method 1 at 90 volts for 3.5 seconds, the purity of the resultant haloperidol-aerosol particles was determined to be 96%. 1.69 mg of aerosol particles were collected for a percent yield of the aerosol of 60%. When 2.1 mg of haloperidol was used and the system was flushed with argon prior to volatilization, the purity of the haloperidol-aerosol particles was determined to be 97%. The percent yield of the aerosol was 29%.

### Example 8

### Loxapine

Loxapine (MW 328, melting point 110° C., oral dose 30 mg), an antipsychotic, was coated on a stainless steel cylinder (8 cm²) according to Method 2. See Example 4. 7.69 mg of loxapine was applied to the substrate, for a calculated loxapine film thickness of 9.2 μm. The substrate was heated as described in Method 2 by charging the capacitors to 20.5 volts. The purity of the loxapine-aerosol particles was determined to be 99.7%. 3.82 mg was recovered from the filter after vaporization, for a percent yield of 50%. A total mass of 6.89 mg was recovered from the test apparatus and substrate, for a total recovery of 89.6%.

### Example 9

### Olanzapine

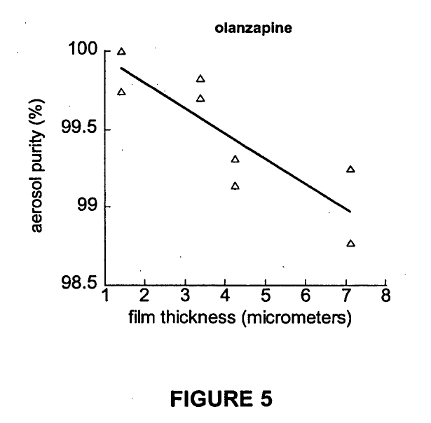

Olanzapine (MW 312, melting point 195° C., oral dose 10 mg), an antipsychotic, was coated onto eight stainless steel cylinder substrates (8-9 cm²) according to Method 2. See Example 4. The calculated thickness of the olanzapine film on each substrate ranged from about 1.2 μm to about 7.1 μm. The substrates were heated as described in Method 2 by charging the capacitors to 20.5 volts. The purity of the olanzapine-aerosol particles from each substrate was determined and the results are shown in FIG. 5. The substrate having a thickness of 3.4 μm was prepared by depositing 2.9 mg of olanzapine. After volatilization of olanzapine from this substrate by charging the capacitors to 20.5 volts, 1.633 mg was recovered from the filter, for a percent yield of 54.6%. The purity of the olanzapine aerosol recovered from the filter was found to be 99.8%. The total mass was recovered from the test apparatus and substrate, for a total recovery of ˜100%. High speed photographs were taken as the olanzapine-coated substrate was heated to monitor visually formation of a thermal vapor. The photographs showed that a thermal vapor was initially visible 30 milliseconds after heating was initiated, with the majority of the thermal vapor formed by 80 milliseconds. Generation of the thermal vapor was complete by 130 milliseconds.

Olanzapine was also coated on an aluminum foil substrate (24.5 cm 2) according to Method 3. See Example 4. 11.3 mg of olanzapine was applied to the substrate, for a calculated thickness of the olanzapine film of 4.61 μm. The substrate was heated as described in Method 3 at 90 volts for 6 seconds. The purity of the olanzapine-aerosol particles was determined to be >99%. 7.1 mg was collected, for a percent yield of 62.8%.

### Example 10

### Prochlorperazine

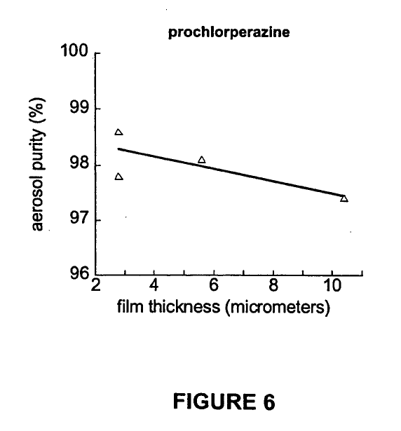

Prochlorperazine free base (MW 374, melting point 60° C., oral dose 5 mg), an antipsychotic, was coated onto four stainless steel foil substrates (5 cm2) according to Method 4. See Example 4. The calculated thickness of the prochlorperazine film on each substrate ranged from about 2.3 μm to about 10.1 μm. The substrates were heated as described in Method 4 by charging the capacitors to 15 volts. Purity of the prochlorperazine-aerosol particles from each substrate was determined and the results are shown in FIG. 6.

Prochlorperazine was also coated on a stainless steel cylinder (8 cm²) according to Method 2. See Example 4. 1.031 mg of prochlorperazine was applied to the substrate, for a calculated prochlorperazine film thickness of 1.0 μm. The substrate was heated as described in Method 2 by charging the capacitors to 19 volts. The purity of the prochlorperazine-aerosol particles was determined to be 98.7%. 0.592 mg was recovered from the filter after vaporization, for a percent yield of 57.4%. A total mass of 1.031 mg was recovered from the test apparatus and substrate, for a total recovery of 100%.

### Example 11

### Promazine

Promazine (MW 284, melting point <25° C., oral dose 25 mg), an antipsychotic, was coated on a piece of aluminum foil (20 cm 2) according to Method 1. See Example 4. The calculated thickness of the promazine film was 5.3 μm. The substrate was heated as described in Method 1 at 90 volts for 5 seconds. The purity of the promazine-aerosol particles was determined to be 94%. 10.45 mg was recovered from the glass tube walls after vaporization, for a percent yield of 99.5%.

### Example 12

### Promethazine

Promethazine (MW 284, melting point 60° C., oral dose 12.5 mg), an antipsychotic, was coated on an aluminum foil substrate (20 cm²) according to Method 1. See Example 4. 5.10 mg of promethazine was applied to the substrate, for a calculated thickness of the promethazine film of 2.6 μm. The substrate was heated as described in Method 1 at 60 volts for 10 seconds. The purity of the promethazine-aerosol particles was determined to be 94.5%. 4.7 mg was recovered from the glass tube walls after vaporization, for a percent yield of 92.2%.

### Example 13

### Quetiapine

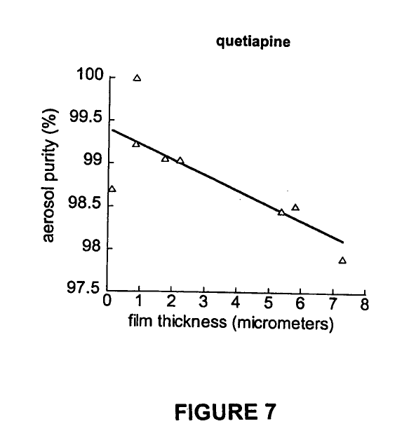

Quetiapine (MW 384, oral dose 75 mg), an antipsychotic, was coated onto eight stainless steel cylinder substrates (8 cm²) according to Method 2. See Example 4. The calculated thickness of the quetiapine film on each substrate ranged from about 0.1 μm to about 7.1 μm. The substrates were heated as described in Method 2 by charging the capacitors to 20.5 volts. Purity of the quetiapine-aerosol particles from each substrate was determined and the results are shown in FIG. 7. The substrate having a quetiapine film thickness of 1.8 μm was prepared by depositing 1.46 mg quetiapine. After volatilization of the quetiapine substrate by charging the capacitors to 20.5 volts, 0.81 mg was recovered from the filter, for a percent yield of 55.5%. The purity of the quetiapine aerosol recovered from the filter was found to be 99.1%. A total mass of 1.24 mg was recovered from the test apparatus and substrate, for a total recovery of 84.9%.

### Example 14

### Trifluoperazine

Trifluoperazine (MW 407, melting point <25° C., oral dose 7.5 mg), an antipsychotic, was coated on a stainless steel cylinder (9 cm²) according to Method 2. See Example 4. 1.034 mg of trifluoperazine was applied to the substrate, for a calculated trifluoperazine film thickness of 1.1 μm. The substrate was heated as described in Method 2 by charging the capacitors to 19 volts. The purity of the trifluoperazine-aerosol particles was determined to be 99.8%. 0.669 mg was recovered from the filter after vaporization, for a percent yield of 64.7%. A total mass of 1.034 mg was recovered from the test apparatus and substrate, for a total recovery of 100%.

Trifluoperazine 2HCl salt (MW 480, melting point 243° C., oral dose 7.5 mg) was coated on a stainless steel cylinder (9 cm²) according to Method 2. Specifically, 0.967 mg of trifluoperazine was applied to the substrate, for a calculated trifluoperazine film thickness of 1.1 μm. The substrate was heated as described in Method 2 by charging the capacitors to 20.5 volts. The purity of the trifluoperazine-aerosol particles was determined to be 87.5%. 0.519 mg was recovered from the filter after vaporization, for a percent yield of 53.7%. A total mass of 0.935 mg was recovered from the test apparatus and substrate, for a total recovery of 96.7%. High speed photographs of trifluoperazine 2HCl were taken as the trifluoperazine-coated substrate was heated to monitor visually formation of a thermal vapor. The photographs showed that a thermal vapor was initially visible 25 milliseconds after heating was initiated, with the majority of the thermal vapor formed by 120 milliseconds. Generation of the thermal vapor was complete by 250 milliseconds.

### Example 15

### Zotepine

Zotepine (MW 332, melting point 91° C., oral dose 25 mg), an antipsychotic, was coated on a stainless steel cylinder (8 cm²) according to Method 2. See Example 4. 0.82 mg of zotepine was applied to the substrate, for a calculated zotepine film thickness of 1 μm. The substrate was heated as described in Method 2 by charging the capacitors to 20.5 volts. The purity of the zotepine-aerosol particles was determined to be 98.3%. 0.72 mg was recovered from the filter after vaporization, for a percent yield of 87.8%. A total mass of 0.82 mg was recovered from the test apparatus and substrate, for a total recovery of 100%. High speed photographs were taken as the zotepine-coated substrate was heated to monitor visually formation of a thermal vapor. The photographs showed that a thermal vapor was initially visible 30 milliseconds after heating was initiated, with the majority of the thermal vapor formed by 60 milliseconds. Generation of the thermal vapor was complete by 110 milliseconds.

### Example 16

### Amoxapine

Amoxapine (MW 314, melting point 176° C., oral dose 25 mg), an anti-psychotic, was coated on a stainless steel cylinder (8 cm²) according to Method 2. See Example 4. 6.61 mg of amoxapine was applied to the substrate, for a calculated amoxapine film thickness of 7.9 μm. The substrate was heated as described in Method D by charging the capacitors to 20.5 volts. The purity of the amoxapine-aerosol particles was determined to be 99.7%. 3.13 mg was recovered from the filter after vaporization, for a percent yield of 47.4%. A total mass of 6.61 mg was recovered from the test apparatus and substrate, for a total recovery of 100%.

### Example 17

### Aripiprazole

Aripiprazole (MW 448, melting point 140° C., oral dose 5 mg), an antipsychotic, was coated on a stainless steel cylinder (8 cm 2) according to Method 2. See Example 4. 1.139 mg of aripiprazole was applied to the substrate, for a calculated aripiprazole film thickness of 1.4 μm. The substrate was heated as described in Method 2 by charging the capacitors to 20.5 volts. The purity of the aripiprazole-aerosol particles was determined to be 91.1%. 0.251 mg was recovered from the filter after vaporization, for a percent yield of 22%. A total mass of 1.12 mg was recovered from the test apparatus and substrate, for a total recovery of 98%. High speed photographs were taken as the aripiprazole-coated substrate was heated to monitor visually formation of a thermal vapor. The photographs showed that a thermal vapor was initially visible 55 milliseconds after heating was initiated, with the majority of the thermal vapor formed by 300 milliseconds. Generation of the thermal vapor was complete by 1250 milliseconds.

A second substrate coated with arirpirazole was prepared for testing. 1.139 mg was coated on a stainless steel cylinder (8 cm 2) according to Method 2, for a calculated aripiprazole film thickness of 1.4 μm. See Example 4. The substrate was heated as described in Method 2 by charging the capacitors to 20.5 volts. The purity of the aripiprazole-aerosol particles was determined to be 86.9%. 0.635 mg was recovered from the filter after vaporization, for a percent yield of 55.8%. A total mass of 1.092 mg was recovered from the test apparatus and substrate, for a total recovery of 95.8%. High speed photographs were taken as the aripiprazole-coated substrate was heated to monitor visually formation of a thermal vapor. The photographs showed that a thermal vapor was initially visible 30 milliseconds after heating was initiated, with the majority of the thermal vapor formed by 200 milliseconds. Generation of the thermal vapor was complete by 425 milliseconds.

### Example 18

### Droperidol

Droperidol (MW 379, melting point 147° C., oral dose 1 mg), an antipsychotic, was coated on a piece of aluminum foil (20 cm²) according to Method 1. See Example 4. The calculated thickness of the droperidol film was 1.1 μm. The substrate was heated according to Method 1 at 90 volts for 3.5 seconds. The purity of the droperidol-aerosol particles was determined to be 51%. 0.27 mg was recovered from the glass tube walls after vaporization, for a percent yield of 12.9%.

Another substrate containing droperidol coated to a film thickness of 1.0 μm was prepared by the same method and heated under an argon atmosphere at 90 volts for 3.5 seconds. The purity of the droperidol-aerosol particles was determined to be 65%. 0.24 mg was recovered from the glass tube walls after vaporization, for a percent yield of 12.6%.

### Example 19

### Fluphenazine

Fluphenazine (MW 438, melting point <25° C., oral dose 1 mg), an antipsychotic, was coated on a piece of aluminum foil (20 cm²) according to Method 1. See Example 4. The calculated thickness of the fluphenazine film was 1.1 μm. The substrate was heated as described in Method 1 at 90 volts for 3.5 seconds. The purity of the fluphenazine-aerosol particles was determined to be 93%. 0.7 mg was recovered from the glass tube walls after vaporization, for a percent yield of 33.3%.

The fluphenazine 2HCl salt form (MW 510, melting point 237° C.) was also tested. Fluphenazine 2HCl was coated on a metal substrate (10 cm²) according to Method 2. See Example 4. The calculated thickness of the Fluphenazine film was 0.8 μm. The substrate was heated as described in Method 2 by charging the capacitors to 20.5 volts. The purity of the fluphenazine 2HCl-aerosol particles was determined to be 80.7%. 0.333 mg was recovered from the filter after vaporization, for a percent yield of 42.6%. A total mass of 0.521 mg was recovered from the test apparatus and substrate, for a total recovery of 66.7%.

### Example 20

### Perphenazine

Perphenazine (MW 404, melting point 100° C., oral dose 2 mg), an antipsychotic, was coated on an aluminum foil substrate (20 cm²) according to Method 1. See Example 4. 2.1 mg of perphenazine was applied to the substrate, for a calculated thickness of the perphenazine film of 1.1 μm. The substrate was heated as described in Method 1 at 90 volts for 3.5 seconds. The purity of the perphenazine-aerosol particles was determined to be 99.1%. 0.37 mg was recovered from the glass tube walls after vaporization, for a percent yield of 17.6%.

### Example 21

### Pimozide

Pimozide (MW 462, melting point 218° C., oral dose 10 mg), an antipsychotic, was coated on a piece of aluminum foil (20 cm²) according to Method 1. See Example 4. The calculated thickness of the pimozide film was 4.9 μm. The substrate was heated as described in Method 1 at 90 volts for 5 seconds. The purity of the pimozide-aerosol particles was determined to be 79%. The percent yield of the aerosol was 6.5%.

### Example 22

### Prochlorperazine 2HCl

Prochlorperazine 2HCl (MW 446, oral dose 5 mg), an antipsychotic, was coated on a stainless steel cylinder (8 cm²) according to Method 2. See Example 4. 0.653 mg of prochlorperazine was applied to the substrate, for a calculated prochlorperazine film thickness of 0.8 μm. The substrate was heated as described in Method 2 by charging the capacitors to 20.5 volts. The purity of the prochlorperazine-aerosol particles was determined to be 72.4%. 0.24 mg was recovered from the filter after vaporization, for a percent yield of 36.8%. A total mass of 0.457 mg was recovered from the test apparatus and substrate, for a total recovery of 70%.

### Example 23

### Risperidone

Risperidone (MW 410, melting point 170° C., oral dose 2 mg), an antipsychotic, was coated on a piece of aluminum foil (20 cm²) according to Method 1. See Example 4. The calculated thickness of the risperidone film was 1.4 μm. The substrate was heated as described in Method 1 at 90 volts for 3.5 seconds. The purity of the risperidone-aerosol particles was determined to be 79%. The percent yield of the aerosol was 7.9%.

Risperidone was also coated on a stainless steel cylinder (8 cm²). 0.75 mg of risperidone was manually applied to the substrate, for a calculated risperidone film thickness of 0.9 μm. The substrate was heated as described in Method 1 by charging the capacitors to 20.5 volts. The purity of the risperidone-aerosol particles was determined to be 87.3%. The percent yield of aerosol particles was 36.7%. A total mass of 0.44 mg was recovered from the test apparatus and substrate, for a total recovery of 59.5%.

### Example 24

### Thiothixene

Thiothixene (MW 444, melting point 149° C., oral dose 10 mg), an antipsychotic, was coated on a piece of aluminum foil (20 cm²) according to Method 1. See Example 4. The calculated thickness of the thiothixene film was 1.3 μm. The substrate was heated as described in Method 1 at 90 volts for 3.5 seconds. The purity of the thiothixene-aerosol particles was determined to be 74.0%. 1.25 mg was recovered from the glass tube walls after vaporization, for a percent yield of 48.1%.

### Example 25

### Ziprasidone

Ziprasidone (MW 413, oral dose 20 mg), an antipsychotic, was coated on a stainless steel cylinder (8 cm²) according to Method 2. See Example 4. 0.74 mg of ziprasidone was applied to the substrate, for a calculated ziprasidone film thickness of 0.9 μm. The substrate was heated as described in Method 2 by charging the capacitors to 20.5 volts. The purity of the ziprasidone-aerosol particles was determined to be 87.3%. 0.28 mg was recovered from the filter after vaporization, for a percent yield of 37.8%. A total mass of 0.44 mg was recovered from the test apparatus and substrate, for a total recovery of 59.5%.

What is claimed is:

1. A method of treating a headache comprising administering by inhalation a composition comprising an antipsychotic to a patient in need of headache relief.

2. The method of claim 1, wherein the peak plasma concentration of the antipsychotic in the patient is obtained within 15 minutes of initiation of inhalation.

3. The method of claim 1, wherein a therapeutic systemic concentration of the antipsychotic in the patient is obtained within 15 minutes of initiation of inhalation.

4. The method of claim 1, wherein the concentration of antipsychotic in the plasma of the patient is at least 30 percent of the peak plasma concentration within 2 minutes of initiation of inhalation.

5. The method of claim 1, wherein headache relief is statistically significant compared to baseline at a time point 15 minutes or less following initiation of inhalation.

6. The method of claim 1, wherein headache relief is statistically significant compared to baseline at a time point 2 hours or less following initiation of inhalation and at a time point 12 hours or more following initiation of inhalation.

7. The method of claim 1, wherein headache severity is decreased at a time point 5 minutes or less following initiation of inhalation.

8. The method of claim 1, wherein headache severity is decreased at a time point 15 minutes or less following initiation of inhalation.

9. The method of claim 1, wherein headache severity is decreased at a time point 30 minutes or less following initiation of inhalation and at a time point 4 hours or more following initiation of inhalation.

10. The method of claim 1, wherein headache severity is decreased at a time point 2 hours or less following initiation of inhalation and at a time point 12 hours or more following initiation of inhalation.

11. The method of claim 1, wherein the patient is headache free at a time point 15 minutes or less following initiation of inhalation.

12. The method of claim 1, wherein the patient is headache free at a time point 2 hours or less following initiation of inhalation and at a time point 12 hours or more following inhalation.

13. The method of claim 1, wherein the mass median aerodynamic diameter of the inhaled composition is about 1 micron to 3 microns.

14. The method of claim 1, wherein the antipsychotic is a non-phenothiazine antipsychotic.

15. The method of claim 1, wherein the non-phenothiazine antipsychotic is selected from haloperidol, droperidol, chlorprothixene, thiothixene, loxapine, molindone, pimozide, flupenthixol, zuclopenthixol, and melperone.

16. The method of claim 1, wherein the antipsychotic is a phenothiazine antipsychotic.

17. The method of claim 16, wherein the phenothiazine antipsychotic is selected from prochlorperazine, trifluoperazine, fluphenazine, promethazine, perphenazine, chlorpromazine, thioridazine, mesoridazine, and acetophenazine.

18. The method of claim 17, wherein the phenothiazine antipsychotic is about 1 mg to 18 mg prochlorperazine.

19. The method of claim 17, wherein the phenothiazine antipsychotic is about 1 mg to 9 mg prochlorperazine.

20. The method of claim 17, wherein the phenothiazine antipsychotic is about 1 mg to 5 mg prochlorperazine.

21. The method of claim 1, wherein the patient self-administers one or more doses of the antipsychotic.

22. The method of claim 21, wherein the patient self-administers a first dose of the antipsychotic, assesses relief after a given interval of time, and, if sufficient headache relief is not obtained, self-administers one or more additional doses.

23. The method of claim 21, wherein the first dose is about 1 mg to 18 mg of the antipsychotic, and wherein the one or more additional doses is about 1 mg to 18 mg of the antipsychotic.

24. A method of treating a headache, comprising administering by inhalation about 1 mg to 18 mg prochlorperazine to a patient in need of headache relief, wherein the prochlorperazine is administered such that the peak plasma concentration of the prochlorperazine is obtained within 15 minutes of initiation of administration of the prochlorperazine and wherein a decrease in headache severity is obtained within 2 hours of prochlorperazine administration.

25. The method of claim 24, wherein the decrease in headache severity persists for at least 12 hours.

26. The method of claim 24, wherein the headache is at least one of a migraine headache, a tension-type headache, or a cluster headache.

27. A method of treating a migraine headache, comprising administering less than 9 mg of an antipsychotic to a patient in need of headache relief, wherein the peak plasma concentration of the antipsychotic is obtained within 15 minutes of initiation of administration of the antipsychotic, wherein a decrease in headache severity is obtained within 1 hour of initiation of administration of the antipsychotic, and wherein the decrease in headache severity persists for at least 12 hours after initiation of administration of the antipsychotic.

28. The method of claim 27, wherein the antipsychotic is prochlorperazine.

29. The method of claim 28, wherein less than 6 mg of prochlorperazine is administered.

30. The method of claim 29, wherein the administration is via inhalation.

31. The method of claim 30, wherein the inhalation is of a condensation aerosol comprising the prochlorperazine.

32. A kit for the treatment of headache comprising an antipsychotic and an inhalation delivery device.

33. The kit of claim 32, wherein the antipsychotic is a phenothiazine antipsychotic.

34. The kit of claim 33, wherein the phenothiazine antipsychotic is selected from prochlorperazine, trifluoperazine, fluphenazine, promethazine, perphenazine, chlorpromazine, thioridazine, mesoridazine, and acetophenazine.

35. The kit of claim 34, wherein the phenothiazine antipsychotic is about 1 mg to 18 mg prochlorperazine.

36. The kit of claim 34, wherein more than one dose of phenothiazine antipsychotic is provided.

37. The kit of claim 32, further including instructions for use.

38. The kit of claim 32, wherein the inhalation delivery device produces a condensation aerosol.
